(19) United States
(12) Patent Application Publication
(10) Pub. No.: US 2004/0253639 A1
(43) Pub. Date: Dec. 16, 2004

(54) Microorganism implicated in intestinal diseases, alzheimer's disease and other diseases

(76) Inventor: John I. Antonius, Fairview, OR (US)

Correspondence Address: GRAYBEAL, JACKSON, HALEY LLP, 155 - 108TH AVENUE NE, SUITE 350, BELLEVUE, WA, 98004-5901, US

(21) Appl. No.: 10/787,860

(22) Filed: Feb. 25, 2004

Related U.S. Application Data
(63) Continuation-in-part of application No. 10/374,455, filed on Feb. 25, 2003.

(51) Int. Cl. G01N 33/53; G06F 19/00; G01N 33/48; G01N 33/50

(52) U.S. Cl. 435/7.1; 702/19

(57) ABSTRACT

A novel microorganism identified as MJA, or MJA, and kits, methods and the like related thereto. The microorganism is implicated in a variety of diseases including Alzheimer's disease, rheumatoid arthritis, ulcers, colitis, IBD, irritable bowel syndrome (IBS), diabetes, atherosclerosis, hypertension, seborrheic keratosis, and cancer.

### CROSS-REFERENCE TO RELATED APPLICATIONS

The present application is a continuation-in-part of U.S. patent application Ser. No. 10/374,455, filed Feb. 25, 2003, which is incorporated herein by reference in its entirety and for all its teachings and disclosures.

### BACKGROUND

Diseases of the human gastrointestinal system cause untold misery to millions of people, as well as massive expense to society at large. For example, according to the National Digestive Diseases Information Clearinghouse (NIDDK) of the National Institutes of Health (NIH), “digestive diseases cost nearly $107 billion in direct health care expenditures in 1992 . . . [including] diarrheal infections ($4.7 billion); gallbladder disease ($4.5 billion); colorectal cancer ($4.5 billion); liver disease ($3.2 billion); and peptic ulcer disease ($2.5 billion).” http://www.niddk.nih.gov/health/digest/pubs/overview/overview.htm#5. The market for sales of anti-ulcerants, alone, in the 12 month period ending March 2002 was $17.8 billion with an annual growth rate of 12%. IMS HEALTH, Drug Monitor, http://www.imshealth.com/public/structure/dispcontent/1,2779,1039-1039-144111,00.html.

As another example, Alzheimer's disease (AD) reportedly cost American businesses $61 billion in 2002, including $25 billion for health care and health care research. The costs appear to be skyrocketing, since they increased from $33 billion in 1998. http://www.alzheimersupport.com/library/showarticle.cfm/ID/1754/e/1/T/Alzheimers/.

The search for and discovery of microorganisms that might be related to such diseases has been the subject of great interest. For example, a search of U.S. Patent Office records on Jan. 15, 2003 for “pylori” (for the microorganism Helicobacter pylori, which was discovered in 1982 and may be related to ulcers, gastritis and possibly some forms of gastric cancer) found that “pylori” was mentioned in 1327 issued patents, and recited in the claims of 293 patents.

Thus, there has gone unmet a need for the identification of microorganisms that are found in or associated with diseases in humans. The present systems and methods provide this and other advantages.

### SUMMARY

The present invention comprises a microorganism that is found in relation to, and implicated in, certain diseases in humans. This microorganism is referred to herein as Microorganism John Antonius, or MJA, after its discoverer. MJA is implicated in, either as a direct (causative) or exacerbating agent, of pathologic processes in the gastrointestinal system of patients with colitis and other GI disorders, and in brain and nervous system of patients with Alzheimer's disease and senile dementia. MJA infects and harms the smooth muscle of the cerebral arteries in the leptomeninges and superficial cortex and other tissues of the brain. The smooth vessels are replaced by amyloid, and the blood vessels are occluded by the process. The entire brain and nervous system, including the choroid plexus, can be affected.

MJA has also been found in diseased tissues (and elsewhere) in patients with thrombotic thrombocytopenic purpura, infections of the gall bladder and GI tract, high blood pressure, diseases of the large muscular blood vessels such as the carotid artery, cerebral artery, and coronary arteries, in the myocardium in congestive heart failure, myocarditis, aging and hypertension; in valvular diseases of the heart; in the choroids plexus and pancreatic vessels in diabetes mellitus type I and II; in the gastrointestinal (GI) tract in inflammatory bowel disease, gastroesophygeal reflux disease (GERD), peptic ulcers; in the skin associated with seborrheic keratosis and chronic dermatitis and in allergic and drug-induced dermatitis. MJA has also been found in humans that have other conditions.

The name MJA is interchangeable with, and therefore includes, other specific names for the microorganism that may arise, i.e., other specific identifiers of equivalent import used to identify the microorganism. It appears that MJA may cause or be an exacerbating factor in at least one of these diseases. Accordingly, the discovery of MJA is a significant advance in the research about such diseases, and may be a significant advance in the diagnosis or treatment of the diseases.

The Figures herein show MJA in the brain and blood vessels of the nervous system and in other locations. Cells of the hematopoetic and immune systems can be affected. MJA has been found with the growth of abnormal cells known as cancer. An example is shown herein of one form of cancer - malignant lymphoma, a cancer of lymphocytes.

In one aspect, the present invention can comprise substantially purified MJA, which MJA can be isolated and specifically identified as MJA. In other embodiments, a kit can comprise a vessel containing isolated MJA and a label specifically identifying the MJA, or a vessel containing substantially purified MJA and a label specifically identifying the MJA. The vessel can be a vial, tube, paraffin block, or microscope slide and can comprising acellular culture medium, cell culture medium or tissue sample such as a blood sample, cytology sample, or biopsy sample. The identification label can recite MJA in writing or can indicate the MJA in a code such as a computer scannable bar code.

In another aspect, the present invention can comprise methods of obtaining MJA comprising: a) identifying an animal suspected of being infected with MJA; and, b) withdrawing a sample of tissue from the animal wherein the sample contains MJA. The methods can further comprise specifically identifying MJA from the tissue, substantially purifying the MJA from the tissue, and if desired growing the MJA in a culture medium or other suitable growth substrate. The methods also comprise specifically identifying MJA. For example, a) providing a sample of tissue suspected of containing MJA; b) examining the sample to determine the presence of MJA; and, c) specifically identifying MJA. The methods can further comprise, prior to the identifying, staining the sample with a stain suitable for MJA; the examining can comprise magnifying and the method further can comprise, after the magnifying, creating an image of the MJA. The magnifying can comprise visible light microscopy, fluorescent light microscopy and electron microscopy and the image can comprise a photomicrograph, electronphotomicrograph, visible light photomicrograph, or fluorescence photomicrograph.

In some embodiments, the invention comprises a labeled image wherein the image comprises MJA and a label specifically identifying the image as depicting MJA. The image further can comprise indicators indicating at least one location of MJA in the image. The image can be maintained in a computer memory, can be a digital image, photographic image, video image or other desired image. In further embodiments, the invention comprises a computer memory configured to specifically identify data therein as representing MJA. The data can comprise an image of MJA, at least one label specifically identifying the image as MJA, and at least one written indicator specifically identifying MJA. The computer memory can be a CD, hard disk or floppy disk.

In another aspect, the present invention comprises compositions comprising an isolated antibody specific substantially only for MJA. the isolated antibody can be a human antibody, polyclonal antibody, monoclonal antibody or other form of binding partner as desired. The antibody can be a substantially complete antibody, substantially only an scFab fragment or other suitable fragments as desired. The antibody can be chemically conjugated to a desired moiety such as a marker or solid substrate.

Also provided are compositions configured for use with an animal, the composition comprising a pharmaceutical amount of an antibody suitable for administration to the animal and specific substantially only for MJA, and at least one of a pharmaceutically acceptable carrier, adjuvant, excipient, buffer and diluent. The composition further can comprise a pharmaceutical amount of at least one anti-Alzheimer's disease drug, anti-rheumatoid arthritis drug, anti-colitis drug, anti-IBD drug, anti-diabetes drug, anti-atherosclerosis drug, anti-hypertension drug, anti-seborrheic keratosis drug, and an anti-cancer drug, or a drug against any other disease or disorder in which MJA is involved. Such drugs include drugs to reduce symptoms, such as painkillers and antihistamines.

In another aspect, the present invention comprises assays for the detection of MJA in a sample. The assays can comprise: a) providing an anti-MJA antibody, b) contacting the anti-MJA antibody with the sample under conditions suitable and for a time sufficient for the anti-MJA antibody to bind to MJA present in the sample, to provide an antibody-bound MJA, and c) detecting the antibody-bound MJA. Other assays for the detection of MJA in a sample can comprise a) providing an anti-idiotypic anti-MJA-antibody antibody, b) contacting the anti-idiotypic anti-MJA-antibody with the sample under conditions suitable and for a time sufficient for the anti-idiotypic anti-MJA-antibody to bind to anti-MJA-antibody present in the sample, to provide an antibody-bound anti-MJA-antibody, and c) detecting the antibody-bound anti-MJA-antibody, and therefrom determining whether the sample contains MJA.

Such detection can be effected, for example, via detection of the antibody, which may be itself detectable or bound to a marker or other identifying aid, and therefrom determining whether the sample contains MJA. The assays can further comprise prior to the contacting, obtaining the sample from an animal, and if desired binding the antibody-bound MJA to a solid substrate. The sample can be an unpurified or purified sample, and can be from any desired animal including a human being. The assay can be selected, for example, a countercurrent immuno-electrophoresis (CIEP) assay, a radioimmunoassay, a radioimmunoprecipitation, an enzyme-linked immuno-sorbent assay (ELISA), a dot blot assay, an inhibition or competition assay, a sandwich assay, an immunostick (dip-stick) assays, a simultaneous assay, an immunochromatographic assay, an immunofiltration assay, a latex bead agglutination assay, an immunofluorescent assay, a biosensor assay, and a low-light detection assay.

In a further aspect, the present invention comprises a pharmaceutical amount of at least one of an isolated humanized or fully human monoclonal antibody specific substantially only for MJA for use in the manufacture of a medicament for inhibiting, preventing or treating MJA in an animal.

In still yet another aspect, the present invention comprises methods of manufacturing a medicament able to reduce symptoms associated with MJA in a human patient, comprising combining a pharmaceutical amount of at least one of an isolated humanized or fully human monoclonal antibody specific substantially only for MJA with at least one of a pharmaceutically acceptable carrier, adjuvant, excipient, buffer and diluent. The methods can further comprise combining at least one additional anti-Alzheimer's disease drug, anti-rheumatoid arthritis drug, anti-colitis drug, anti-IBD drug, anti-diabetes drug, anti-atherosclerosis drug, anti-hypertension drug, anti-seborrheic keratosis drug, and anti-cancer drug.

In still another aspect, the present invention comprises pharmaceutical compositions configured to inhibit MJA in an animal such as a human, mammal or farm animal, for example by preventing an MJA infection, reducing the severity or amount of MJA in a patient, or eliminating MJA from a patient. In some embodiments, methods herein comprise administering such compositions to a patient. If desired, the administration can also comprise administering least one additional drug suitable to reduce at least one symptom of a disease in which MJA can be implicated. The at least one additional drug can be at least one of an anti-Alzheimer's disease drug, anti-rheumatoid arthritis drug, anti-colitis drug, anti-IBD drug, anti-diabetes drug, anti-atherosclerosis drug, anti-hypertension drug, anti-seborrheic keratosis drug, and anti-cancer drug. The at least one additional drug and the pharmaceutical composition can be in a single composition or in separate compositions, and can be administered sequentially or simultaneously.

Thus, in some embodiments the present invention provides methods of reducing an amount of MJA in an MJA infection in an animal comprising administering an effective amount of a desired anti-MJA drug to the animal in an amount and for a time sufficient to reduce the amount of the MJA infection, and methods of reducing at least symptom associated with an MJA infection in an animal comprising administering an effective amount of a desired anti-MJA drug to the animal in an amount and for a time sufficient to reduce the at least symptom associated with the MJA infection. Other embodiments comprise methods of treating an animal, comprising determining that the animal has a putative MJA infection, then administering to the animal a pharmaceutical composition as discussed herein.

In some embodiments, the anti-MJA drug can be at least one of doxycycline, tetracycline, quinolone, nitroimidazole, nitrofuran, rifamycin, streptomycin, an aminocyclitol, chloramphenicol, kanamycin, gentamycin, erythromycin, azithromycin, lincomycin, clindamycin, fusidic acid, and mupirocin. Suitable quinolones include norfloxacin, pefloxacin, ciprofloxacin, oflaxacin, ruvioxacin, Ro-091168, KB-5246, 4-quinolone, isothiazolo-quinolone, pyrridoquinolone and nalidixic acid.

In some embodiments, the at least one symptom can be a symptom associated with a gastrointestinal disease such as colitis, inflammatory bowel disease, irritable bowel syndrome, ulcer, Alzheimer's disease, rheumatoid arthritis, diabetes, atherosclerosis, hypertension, seborrheic keratosis, and cancer. Exemplary cancers include squamous cell carcinoma of the tonsil region (the salivary glands had been destroyed by MJA), squamous cell carcinoma of the lung, adenocarcinoma of the endometrium, adenocarcinoma of the colon, oligodendroglioma of the brain, and undifferentiated small cell carcinoma of the lung. Accordingly, in some embodiments the invention can comprise treating one or more of such diseases according to procedures and protocols discussed herein.

The kits can also comprise vessels containing pharmaceutical compositions as discussed herein. The kits can also be for the detection of MJA in a sample and comprise: a) at least one antibody specific substantially only for MJA; b) at least one of a reagent and a device for detecting the antibody; and c) a label stating that the kit can be to be used for the detection of MJA. Such kits can comprise a label specific to MJA, can comprise instructions for use of the composition to at least one of identify MJA and inhibit MJA.

These and other aspects, features and embodiments are set forth within this application, including the following Detailed Description and attached drawings. In addition, various references are set forth herein, including in the Cross-Reference To Related Applications, that discuss in more detail certain results, systems, apparatus, methods and other information; all such references are incorporated herein by reference in their entirety and for all their teachings and disclosures, regardless of where the references may appear in this application. Unless expressly stated otherwise or clear from the context, all embodiments, aspects, features, etc., can be mixed and matched, combined and permuted in any desired manner.

### BRIEF DESCRIPTION OF THE DRAWINGS

FIG. 1 is an electron micrograph of an MJA in a red blood cell (RBC).

FIG. 2 is a 1,000× photomicrograph of MJA in a 1% crystal-violet stained, wet-mounted blood sample from a human with colitis.

FIG. 3 is a 1,000× photomicrograph of MJA in a 1% crystal-violet stained, wet-mounted blood sample from a human with bronchitis and diarrhea.

FIG. 4 is a 1,000× photomicrograph of MJA in a Genta stained colon sample from a human with colitis and polyps of the colon.

FIG. 5 is a 400× photomicrograph of MJA in an H&E stained colon sample from a human with non-specific colitis.

FIG. 6 is a 1,000× photomicrograph of MJA in a Steiner stained colon sample from a human with colitis.

FIG. 7 is a 1,000× photomicrograph of MJA in a Steiner stained vascular muscle of the colon from a human with colitis.

FIG. 8 is a 1,000× photomicrograph of MJA in a preparation stained for electron microscopy. The preparation is a colon sample from a human with colitis.

FIG. 9 is a 400× photomicrograph of MJA in a Steiner stained colon sample from a human with non-specific colitis.

FIG. 10 is a 1,000× photomicrograph of MJA in an H&E stained duodenum sample from a human with abdominal discomfort, fatigue and a general feeling of sickness.

FIG. 11 is a 1,000× photomicrograph of MJA in a Steiner stained polyp from the colon from a human with colitis.

FIG. 12 is a 40× photomicrograph of MJA in a Genta stained colon polyp sample from a human with colitis.

FIG. 13 is a 1,000× photomicrograph of MJA in a Steiner stained male breast sample from a human with gynecomastia.

FIG. 14 is a 1,000× photomicrograph of MJA in an H&E stained salivary gland sample from a human.

FIG. 15 is a 1,000× photomicrograph of MJA in an H&E stained choroid plexus sample from a human that had died from diabetes mellitus.

FIG. 16 is a 1,000× photomicrograph of MJA in a Steiner stained section of yolk sac from a fertilized chicken egg that had been infected with MJA.

FIG. 17 A-J. Brain. These show microorganisms in neurons, glial tissue and blood vessels of the brain. In blood vessels the microorganism is seen in the smooth muscle cells of the media, in the adventitia, the endothelium and in the blood and red blood cells. From a 77-year-old man who died from Alzheimer's disease.

FIG. 18A-E. Gastrointestinal Tract.

FIG. 19A & B. Malignant lymphoma.

FIG. 20A, B & C. Prostate.

FIG. 21. Breast Disease.

FIG. 22A & B. Myocardium.

FIG. 23A & B. Lung.

FIG. 24A-D. Carotid artery.

FIG. 25 is an electron micrograph of an MJA in a red blood cell (RBC).

DETAILED DESCRIPTION

Illnesses such as Alzheimer's disease, arterial disease, colitis and inflammatory bowel disease (IBD) cause much human suffering. The search for the cause(s) of, and other factors in, such diseases is ongoing. A newly discovered microorganism, MJA, has been found in humans that have these, and other, conditions. MJA may exacerbate or cause one or more of such diseases or conditions. Thus, the discovery of MJA is a significant advance in research about such diseases, and may also be a significant advance in the diagnosis or treatment of the diseases or conditions.

### Definitions

All terms used herein, including those specifically discussed in this section, are used in accordance with their ordinary meanings unless the context or definition clearly indicates otherwise. Also unless indicated otherwise, except within the claims, the use of “or” includes “and” and vice-versa. Non-limiting terms are not to be construed as limiting unless expressly stated, or the context clearly indicates, otherwise (for example, “including,” “having,” and “comprising” typically indicate “including without limitation”). Singular forms, including in the claims, such as “a,” “an,” and “the” include the plural reference unless expressly stated, or the context clearly indicates, otherwise.

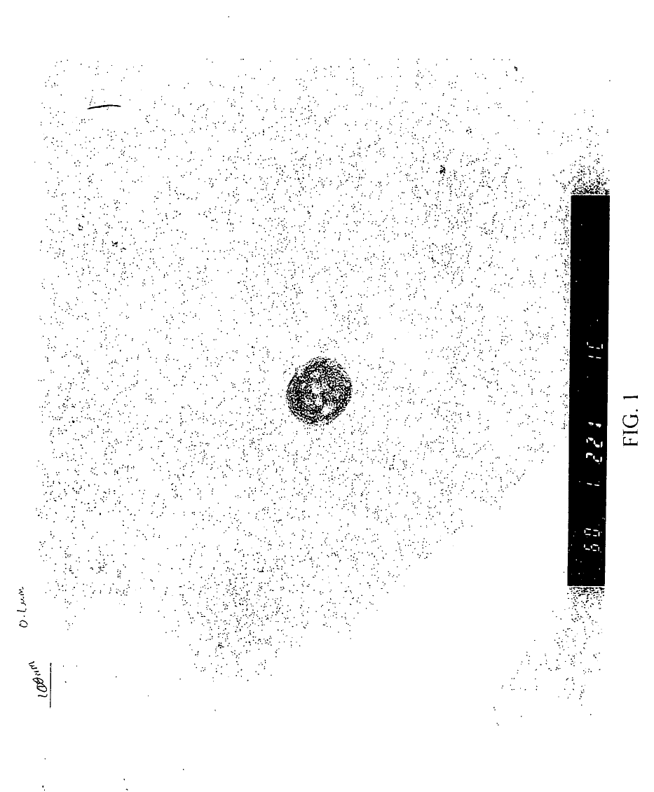

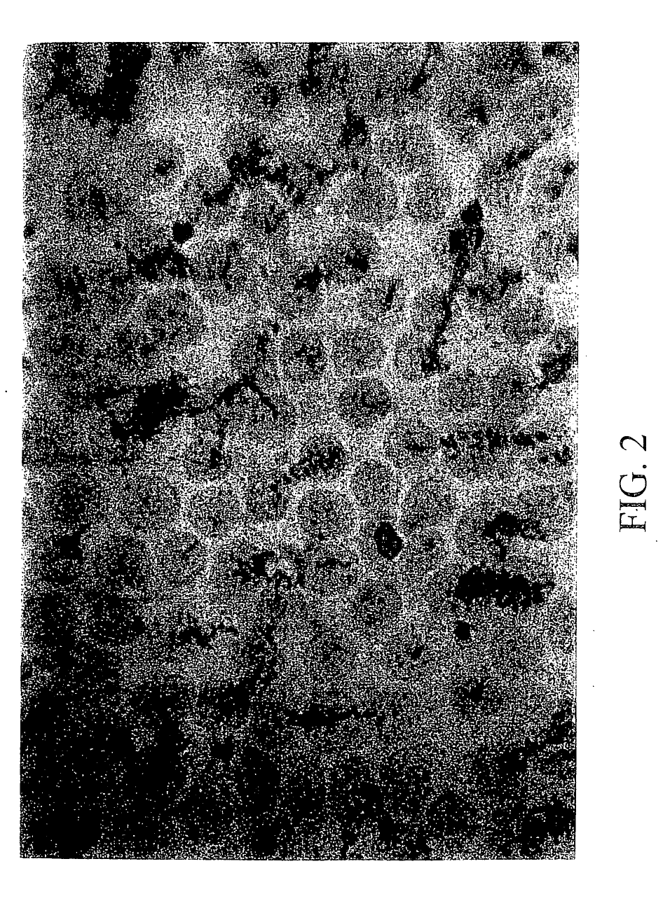

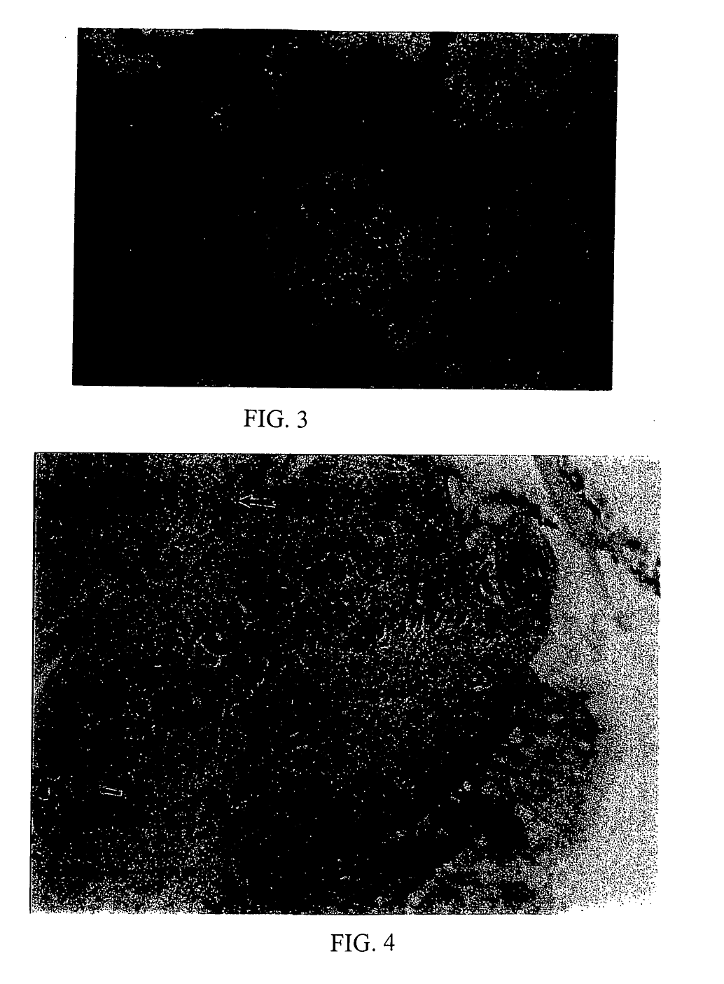

“Microorganism John Antonius (MJA)” is a specific identifier for the microorganism that is the subject of this application and is interchangeable with, and therefore includes, other specific names for the microorganism that may arise, i.e., other specific identifiers of equivalent import used to identify the microorganism, and also includes mutants, recombinants and variants of MJA. Examples of MJA are shown in the electron micrograph of FIG. 1 and in the photomicrographs of FIGS. 2-16. MJA was deposited in February, 2003, with the American Type Culture Collection (ATCC), 10801 University Boulevard, Manassas, Va., U.S.A. 20110-2209, under the provisions of the Budapest Treaty for the International Recognition of the Deposit of Microorganisms for the Purposes of Patent Procedure and was accorded ATCC Accession No. PTA5003. The deposit is provided as a convenience to interested parties, and is not an admission that a deposit is required. A license may be required to make, use, or sell the deposited materials; no such license is granted hereby.

“Antagonist” refers to a molecule which interacts with MJA, for example by binding to MJA, and prevents, inactivates, decreases or shortens the amount or the duration of the effect of the biological activity of MJA. Antagonists include anti-MJA antibodies and other MJA specific binding partners, or any other molecules that so affect MJA. Antagonists and other modulators of MJA can be identified using in vitro or in vivo assays for MJA-mediated signaling. Samples or assays comprising MJA that are treated with a potential antagonist are compared to control samples without the antagonist to examine the extent of inhibition. Control samples can be assigned a relative MJA activity value of 100%. Antagonist activity on MJA is achieved when MJA activity value relative to the control is at most about 90%, typically about 80%, optionally about 50% or about 25 to 0% of the 100% value.

“Antibody” indicates one type of binding partner, typically encoded by an immunoglobulin gene or genes, and refers to, for example, intact monoclonal antibodies, polyclonal antibodies, phage display antibodies, and multispecific antibodies (e.g., bispecific antibodies) formed, for example, from at least two intact antibodies. Antibody also refers to fragments thereof, which comprise a portion of an intact antibody, generally the antigen-binding or variable region of the intact antibody that is capable of binding the epitopic determinant. Examples of antibody fragments include scFv, Fab, Fab′, F(ab′)₂, and Fv fragments, diabodies, linear antibodies, single-chain antibody molecules, and multispecific antibodies formed from antibody fragments. See U.S. Pat. No. 6,214,984. Antibody fragments may be synthesized by digestion of an intact antibody or synthesized de novo either chemically or utilizing recombinant DNA technology. Antibodies according to the present invention have at least one of adequate specificity, affinity and capacity to perform the activities desired for the antibodies. Antibodies can, for example, be monoclonal, polyclonal, or combinatorial. Antibodies that bind MJA polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

“Antigenic determinant” refers to the antigen recognition site on an antigen (i.e., epitope). Such antigenic determinant may also be immunogenic.

“Composition” indicates a combination of multiple substances into a mixture.

“Gene” refers to the basic unit of heredity that carries the genetic information for a given RNA or protein molecule. A gene is composed of a contiguous stretch of DNA and contains a coding region that is flanked on each end by regions that are transcribed but not translated. A gene is a segment of DNA involved in producing a biologically active polypeptide chain.

“Humanized antibody” refers to antibody molecules in which the amino acid sequence in the non-antigen-binding regions has been altered so that the antibody more closely resembles a human antibody, and still retains its original binding ability. For example, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are typically made to further refine and optimize antibody performance. In some embodiments, the humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin sequence. The humanized antibody optimally also will comprise at least a portion of an immunoglobulin constant region (Fc), typically that of a human immunoglobulin. For further details see, e.g., Jones et al., Nature, 321:522-525 (1986); Reichmann et al., Nature, 332:323-329 (1988); and, Presta, Curr. Op. Struct. Biol., 2:593-596 (1992).

“Immune response” refers to any of the body's immunologic reactions to an antigen such as antibody formation, cellular immunity, hypersensitivity, or immunological tolerance.

“Isolated” generally means that the material is removed from its original environment (e.g., its natural environment if it is naturally occurring). When referring to MJA, isolated means that the MJA has been separated from its host, for example in a tissue sample, a pure tissue culture, etc. Thus, a naturally-occurring MJA in a living animal is not isolated. But the same MJA, separated by artificial methods from the animal is isolated.

“Microarray” refers to an array of distinct nucleic acid or amino acid molecules arrayed on a substrate, such as paper, nylon or any other type of membrane, filter, chip, glass slide, or any other suitable solid support. Microarrays can also refer to tissue microarrays, composed of small tissue pieces arranged on a slide. U.S. Pat. No. 5,143,854 and PCT Patent Publication Nos. WO 90/15070 and 92/10092.

“Modulate” refers to controllably changing the activity of a substance or other item, such as the biological activity of MJA. In the present application, typically, MJA is down modulated via the action of an anti-MJA antibody.

“Monoclonal antibody” refers to an antibody obtained from a population of substantially homogeneous antibodies, e.g., the individual antibodies comprising the population are identical except for possible naturally occurring mutations that may be present in minor amounts. Monoclonal antibodies include “chimeric” antibodies (immunoglobulins) in which a portion of the heavy or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. U.S. Pat. No. 4,816,567; Morrison et al., P.N.A.S. USA, 81:6851-6855 (1984). Monoclonal antibodies are highly specific, being directed against a single antigenic site. As a matter of distinction, polyclonal antibody preparations typically include different antibodies directed against different determinants (epitopes) of a target antigen whereas each monoclonal antibody is directed against a single determinant on the antigen. Monoclonal antibodies can be synthesized by hybridoma culture, uncontaminated by other immunoglobulins. For example, the monoclonal antibodies to be used in accordance with the present invention may be made by the hybridoma method first discussed by Kohler and Milstein, Nature, 256:495 (1975), or may be made by recombinant DNA methods. See, e.g., U.S. Pat. No. 4,816,567. Monoclonal antibodies may also be isolated from phage antibody libraries using the techniques discussed in Clackson et al., Nature, 352:624-628 (1991), and Marks et al., J. Mol. Biol., 222:581-597 (1991), for example. The modifier “monoclonal” indicates the character of the antibody as being obtained from a substantially homogeneous population of antibodies, and is not to be construed as requiring production of the antibody by any particular method.

“Sample” is used in its usual broad sense. For example, a biological sample suspected of containing MJA may comprise a bodily fluid, tissue, tissue print, and the like. Biological sample refers to samples from a healthy individual as well as to samples, for example, from a subject having or suspected of having MJA or a disease in which MJA is implicated. Examples of such diseases include Alzheimer's disease, rheumatoid arthritis, ulcers, colitis, IBD, irritable bowel syndrome (IBS), diabetes, atherosclerosis, hypertension, seborrheic keratosis, and cancer, or any other disease or disorder in which MJA is involved.

“Specific binding” or “specifically binding” refers to an interaction between a protein or peptide and a certain substance, such an antibody specific for the protein. The interaction is dependent upon the presence of a particular structure of the protein recognized by the binding molecule (e.g., the antigenic determinant or epitope). For example, if an antibody specifically binds epitope “A,” the presence of a polypeptide containing epitope A or the presence of free unlabeled epitope A will reduce the amount of labeled epitope A that binds to the antibody in a reaction containing free labeled epitope A and the antibody. Conversely, the presence of a polypeptide that does not contain epitope A will not reduce the amount of labeled epitope A that binds to the antibody. Highly specific binding indicates that the antibody, etc., does not bind in a significant amount to other proteins present in the sample. Typically, a specific or selective reaction will be at least twice the background signal or noise and more typically more than 10 to 100 times or more the background signal or noise.

“Substantially purified” refers to MJA removed from its natural environment and separated from a significant portion of the other components of such natural environment. Highly purified indicates that substantially all other components of its natural environment have been removed, for example in an acellular culture medium or in a tissue culture where the tissue is not from the original host of the particular MJA (such as a commercially available tissue or cellular culture medium). Purity can be assayed by standard methods using traditional techniques that are readily apparent to a skilled person in view of the present application.

The scope of the present discussion includes both means plus function and step plus function concepts. However, the terms set forth in this application are not to be interpreted in the claims as indicating a “means plus function” relationship unless the word “means” is specifically recited in a claim, and are to be interpreted in the claims as indicating a “means plus function” relationship where the word “means” is specifically recited in a claim. Similarly, the terms set forth in this application are not to be interpreted in method or process claims as indicating a “step plus function” relationship unless the word “step” is specifically recited in the claims, and are to be interpreted in the claims as indicating a “step plus function” relationship where the word “step” is specifically recited in a claim.

### The Discovery of MJA.

While reviewing biopsies from two patients while working in Saudi Arabia, the inventor (a pathologist) saw unidentifiable microorganisms in the tissues. He could see the microorganisms in electron microscopy and micrographs, but could not find them using ordinary investigative procedures such as growing them in culture or using sero-immunological studies using known antibodies against known organisms.

Later, in another laboratory, the inventor reviewed stomach and gastrointestinal tract biopsies of a number of gastrointestinal patients that he could not classify into a specific disease class such as Crohn's disease or ulcerative colitis. In the gastrointestinal tissues of these cases he discovered a microorganism in the smooth muscle layers in the blood vessels and in the epithelium. He also discovered that the blood vessels were blocked by the unidentified microorganism or something associated with the unidentified microorganism. The blocking material when stained with eosin Y was highly fluorescent under UV light but was not visible under white light nor UV light without the eosin Y staining. Such microorganism appeared to be the same as the unidentified microorganism he had seen previously. The blockages indicated that the microorganism may have access to the bloodstream and thus could possibly infect a variety of organs.

The inventor then launched a study of the unidentified microorganism upon returning to the United States. Among other things, he used atypical stains, fluorescent light and regular and electron microscopes. Using the fluorescence, the inventor observed that blood vessels in the colon of patients (often patients with only mild symptoms) were damaged and blocked to a marked degree. This was important, in part, because most pathologists believed the ulcerations in the colon were like that of other ulcerating diseases, such as typhoid, dysentery and cholera, where the organisms were on the surface. However, the inventor did not find MJA at that time on the surface, or lumen, of the colon, but rather deep in the tissue (MJA is typically difficult to find on the lumen).

Samples from two cases were examined by growing the microorganisms in chick embryo tissue culture. Biopsies from the cases were taken at the time of surgery, the biopsies were placed in sterile physiological saline with 50 μg/ml gentamycin for approximately 2 hours, then rinsed several times in saline. The biopsies were then ground up in 1 ml of physiological saline using a mortar and pestle, the resulting suspension was aspirated into a syringe then 0.2 ml was injected into the yolk sac of 6 day old chick embryos. They were incubated 6 days then harvested. Tissue from the yolk sac membrane was placed in 10% formalin and histologic sections were made and Steiner stained.

The presence of unidentified microorganisms was confirmed, which microorganisms were located in the embryo cells in locations that substantially correlated to the locations in the biopsies from the human patients. These particular cultures were not maintained over time because the embryos were harvested for histological examination. MJA has since been grown in culture using Vitacell RPMI-1640 culture medium with and without 20% fetal calf serum (ATCC) and in the allantoic fluid and other locations of fertilized chicken eggs.

Thus, MJA was discovered, and isolated and substantially purified, from its original tissue.

Some further characterization of MJA.

FIG. 1 is an electron micrograph of an MJA in an RBC.

FIGS. 2 & 3 show 1,000× blood smears with MJA in the RBCs and plasma. The blood smears were prepared as wet mounts of 1-2% Crystal Violet.

FIG. 4 shows a 1,000× histologic section of a Colon biopsy that has been Genta stained. The small arrow shows black aggregates of MJA within lining cells of the colon. The large arrow shows MJA scattered in the stroma. Nuclei of lining cells are not well seen, but the rows of lining cells can be identified.

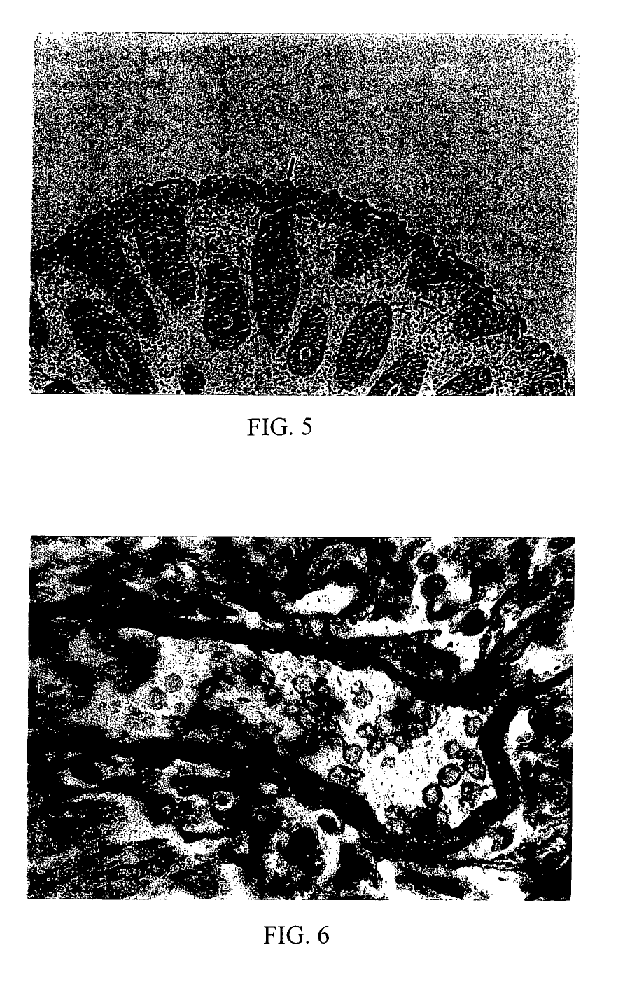

FIG. 5 shows a 400× hemotoxylin and eosin (H&E) stained histologic colon section from a human with non-specific colitis. There is inflammation in the supporting stroma. MJA stained blue. Many of the surface cells are infected, with the MJA in substantially the same stage of development.

FIG. 6 shows a 1,000× Steiner stained small vessel in the colon. The vessel wall contains MJA and the lumen contains RBCs with MJA. MJA is also seen in plasma.

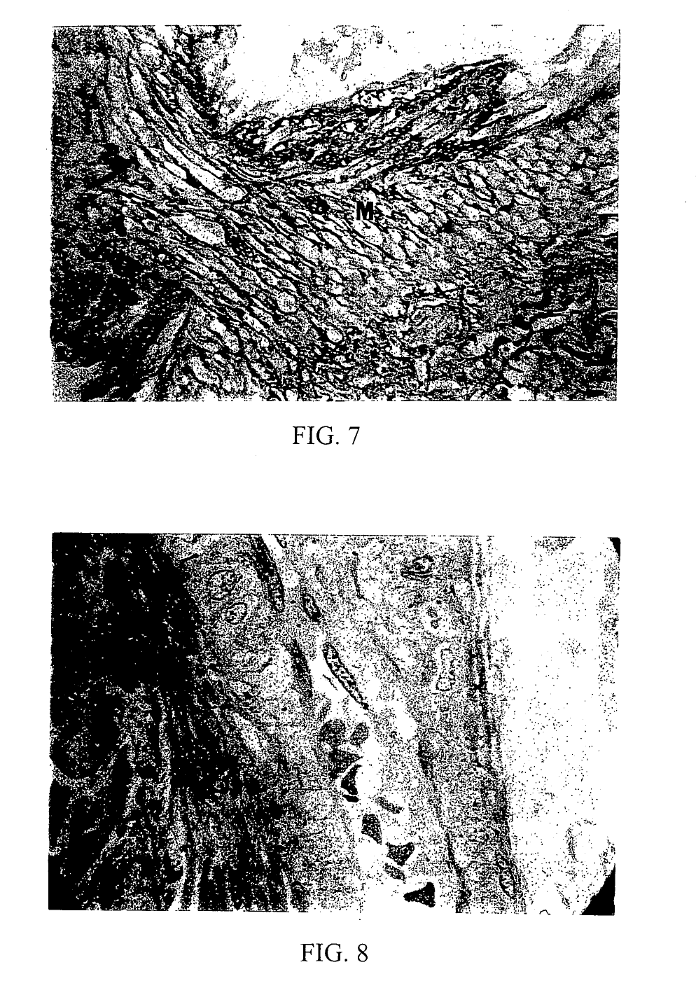

FIG. 7 shows a 1,000× medium sized blood vessel in the colonic muscle with MJA showing as black dots in the muscle.

FIG. 8 shows a 1,000× light photomicrograph thick section of the colon wall embedded in plastic and stained with toluidine blue for E.M. study. The black dots are MJA and can be seen intracellularly and intravascularly.

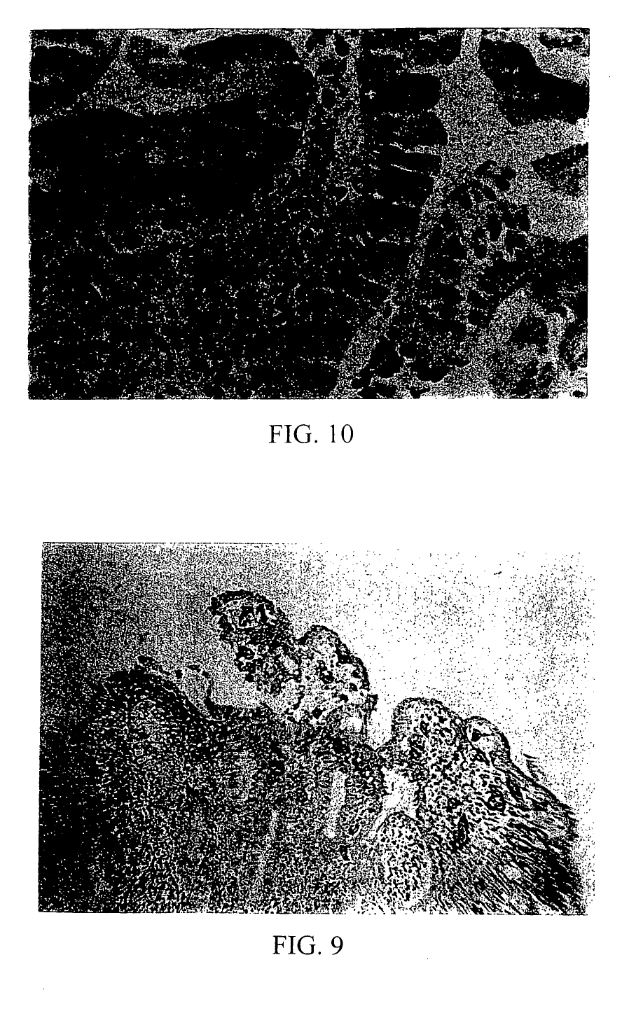

FIG. 9 shows a 400× histologic section of duodenum from a human with colitis. MJA stains black (arrows) and can be seen in small vessels, which are dilated and some are occluded. This pattern of vessels involvement produces swelling, redness and ulceration of the mucosa, which is similar to the pathogenesis of colitis.

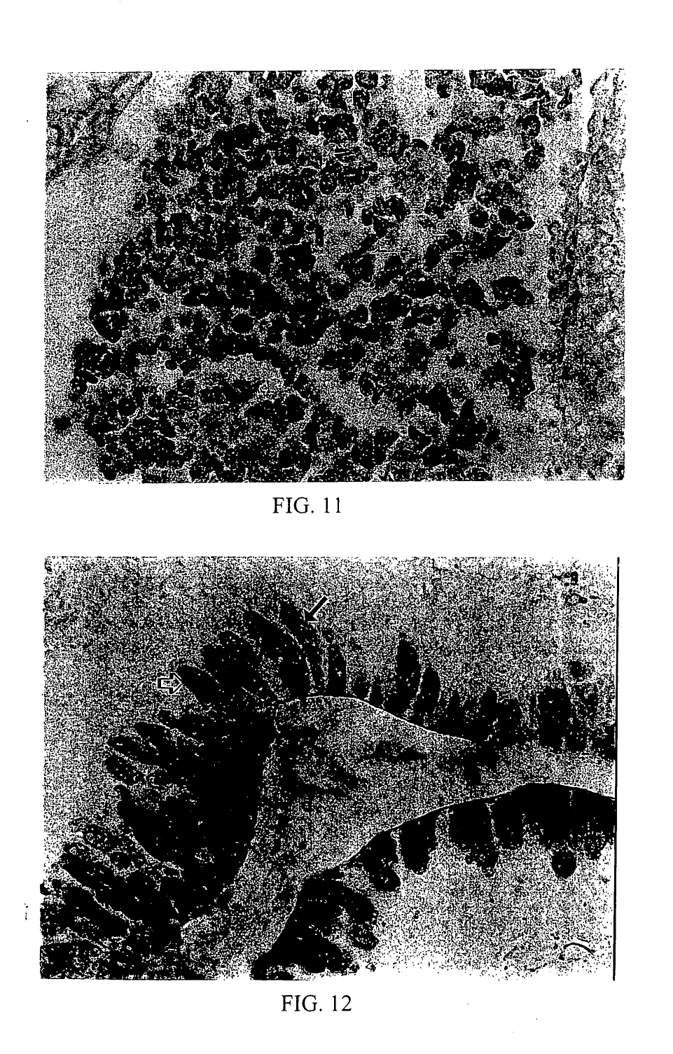

FIG. 10 shows a 1,000× duodenum section from a human with abdominal discomfort, fatigue and a sick feeling The MJA are black and scattered through the stroma, in lymphatics and capillaries. The MJA vary in shape and size.

FIG. 11 shows a 1,000× photomicrograph of a Steiner stained blood vessel of a colon polyp removed by polypectomy. MJA appears as black dots in and on the RBCs in the lumen of the vessel. Larger dots appear to represent groups or clumps of MJA.

FIG. 12 shows a 1,000× Genta stained section of an adenomatous polyp of the colon. MJA stained black and mucus stained blue. The closed arrow shows a cell full of MJA and the open arrow shows a cell with mucus stained blue and MJA stained black.

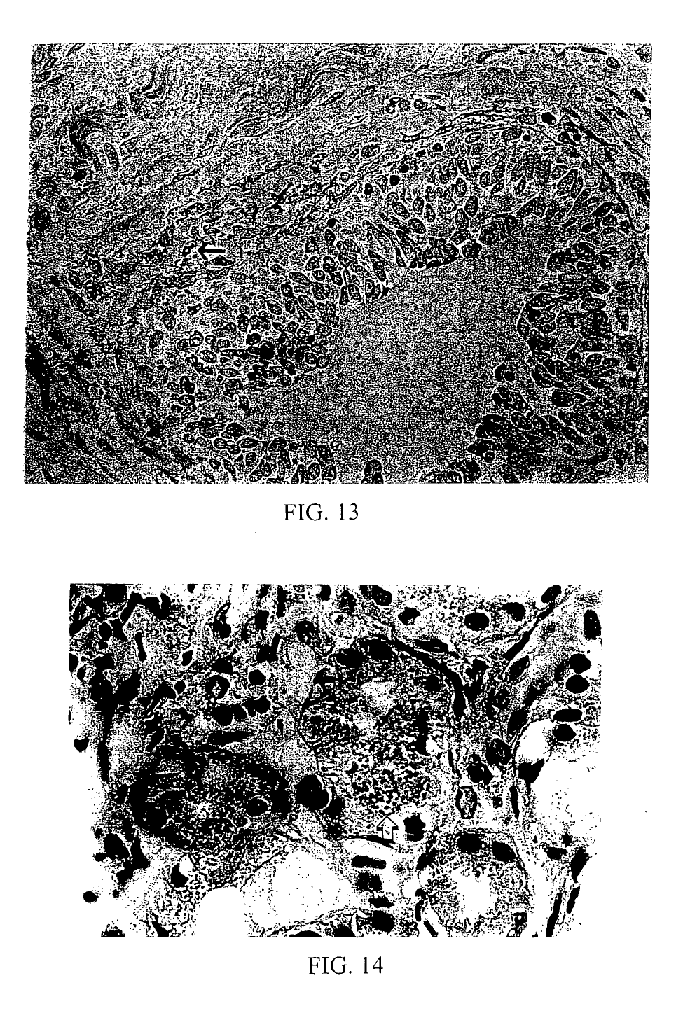

FIG. 13 shows 1000× Steiner stained tissue section from male breast from a human with gynecomastia. The veins and lymphatics around the ducts from male breast (arrow) show MJA, which appear as black dots.

FIG. 14 shows 1000× H&E stained section of salivary gland showing changes in lining cells and round forms of MJA.

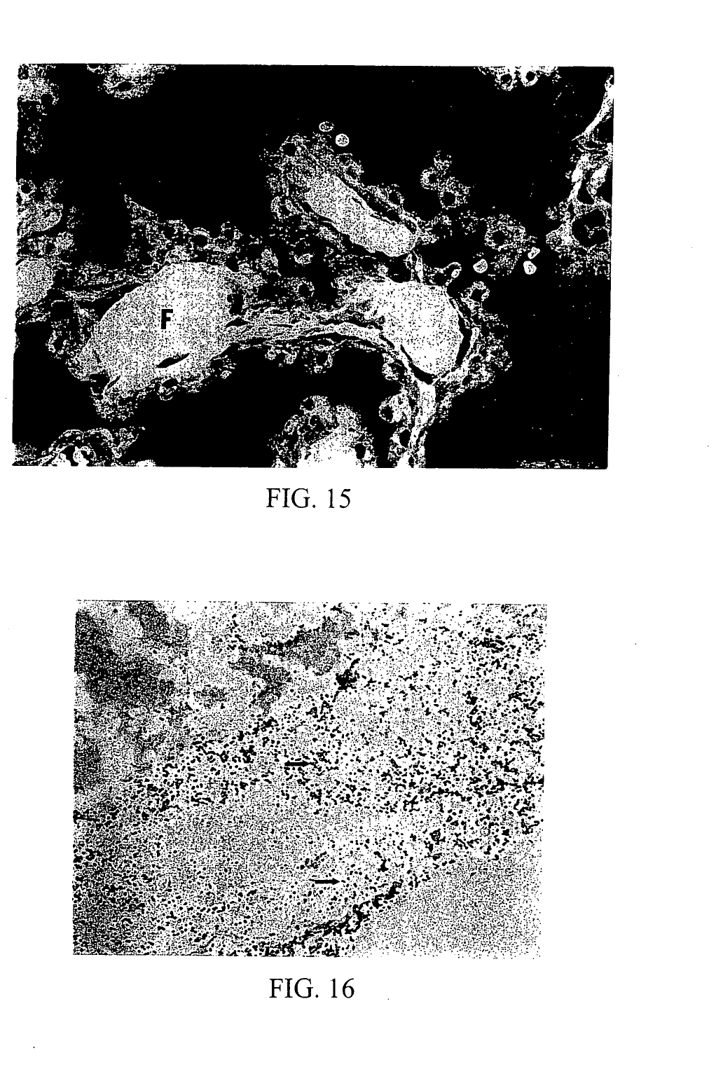

FIG. 15 shows 1000× H&E stained choroid plexus from a human that died at age 37 from diabetes mellitus. The picture was taken under UV light and through a filter that eliminated the excitation light. The lumens of the vessels are occluded by a fluorescent material (F) that appears to be related to MJA.

FIG. 16 shows 1000× Steiner stained histologic section of yolk sac from a fertilized chicken egg that had been infected with MJA. The MJA appear black and vary in size. The arrows point to individual MJA.

Based upon the information in the Figures and other observations, it appears that MJA has a complex life cycle and growth cycle. It can range in size from very small to large, for example from about 0.5 to 2.5 microns, and can aggregate to create a mass in the cell. It can leave cells. MJA can be found in a variety of cells and tissues as well as in interstitial spaces.

In the inventor's hands, MJA did not stain with Wright's stain, but did stain with Giemsa, the Steiner stain and crystal violet stain (MJA stained with crystal violet, but did not retain the stain when subjected to washing in the Gram stain protocol, thus MJA did stain with crystal violet but is also Gram negative). See, e.g., FIGS. 2-16. MJA found in red blood cells (RBCs) did not stain with hematoxylin although MJA showed up as blue dots when stained with hematoxylin in other locations. The reason for this failure to stain with hematoxylin is unknown, but MJA may be retained in vacuoles or other structures in the RBCs that prevent the stain from reaching MJA.

In order to determine whether MJA is potentially antigenic, colon tissue sections from four colitis patients infected with MJA were incubated with serum from the patient's own blood. If the tissue section contained an antigenic, and therefore likely pathogenic, microorganism the antibodies would attach to the microorganism. An immunohistochemical (IHC) test found antibodies bound to the tissue sections at the same sites where MJA was found by silver staining methods, which thus indicates that MJA is antigenic.

MJA has been found in the blood stream, and accumulated in the small vessels. Examples of this can be seen in most of the Figures, as discussed below and elsewhere herein. The levels of carbon dioxide and carbonic acid are high in the small veins, which may provide a positive environment for MJA.

Evidence of the potential pathogenicity of MJA includes the observation that the presence of MJA correlates with vascular blockage. MJA is also found in large numbers in the peritoneal cavity in peritonitis cases. MJA occurs in exudates of inflammatory cells, as do other pathogens. Also, as noted above, MJA is antigenic, which is often indicative of pathogenicity.

Some of the characteristics associated with MJA are consistent with features of various inflammatory and/or diarrheal disorders of the gastrointestinal (GI) tract, such as colitis, inflammatory bowel disease and many gastrointestinal cases with minimal symptoms. For example, early ulcerative colitis features redness of the mucosa with swelling of the mucosal folds and edema of the submucosa. As the disease advances, small hemorrhages and ulcers develop, then the size, wall shapes and motility of the intestine change. These symptoms are typical of small vessel obstruction, which as noted above is a characteristic of MJA. Lesions may be minor if the area is small and small numbers of MJA are involved.

In at least some cases of infection of the gastrointestinal tract, and particularly the small bowel (jejunum and ileum) some absorptive surface cells infected with MJA appear to change to resemble goblet cells. Most of the cells in the mucosal lining of the small bowel are normally absorptive cells; the inventor has observed in some cases over half infected by MJA. MJA can start off very small, then grow larger and can be eventually shed into the bowel lumen. Infected cells begin small, fill up with MJA then extrude the MJA into the bowel lumen.

When multiple biopsies were taken along an infected gastrointestinal tract, each biopsy shows MJA at substantially the same stage of development. FIG. 5 shows an example of such a biopsy, where multiple lining cells contain MJA in the same stage of development. It appears that MJA can divide and grow in synchrony, and such growth can be localized, even to an organ or a part of an organ. This growth pattern accords with the progression of explosive attacks in colitis. For example, a patient can appear to be well then one hour later be prostrate, sweating and immobilized, with diarrhea at one end and vomiting at the other. Bleeding, hemorrhage and shock may result in a surgical emergency or even death. Long-term consequences can include involvement of other organs, aging and development of cancer. During these acute attacks MJA has been found in large numbers in blockages of blood vessels.

### MJA in the Brain—Alzheimer's Disease, Senile Atrophy and Aging

Patient History:

77—year old male with Dementia

Brain weight 1100 gr.

Coronary artery bypass for coronary artery occlusion

Atherosclerosis

Chronic Pneumonia

Absence of Gall Bladder

Fatty liver

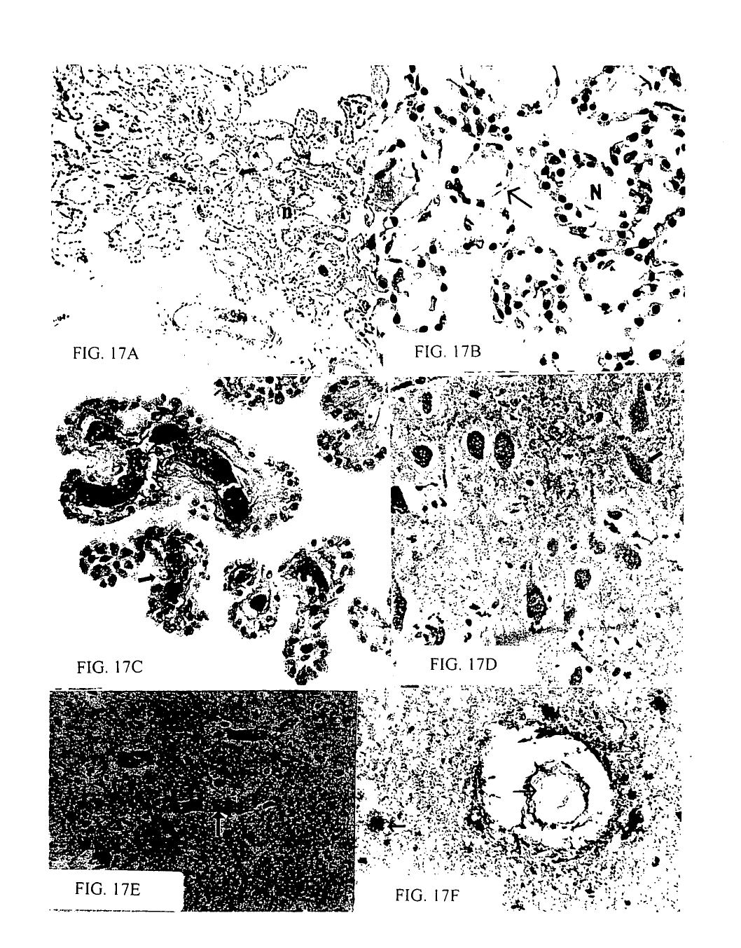

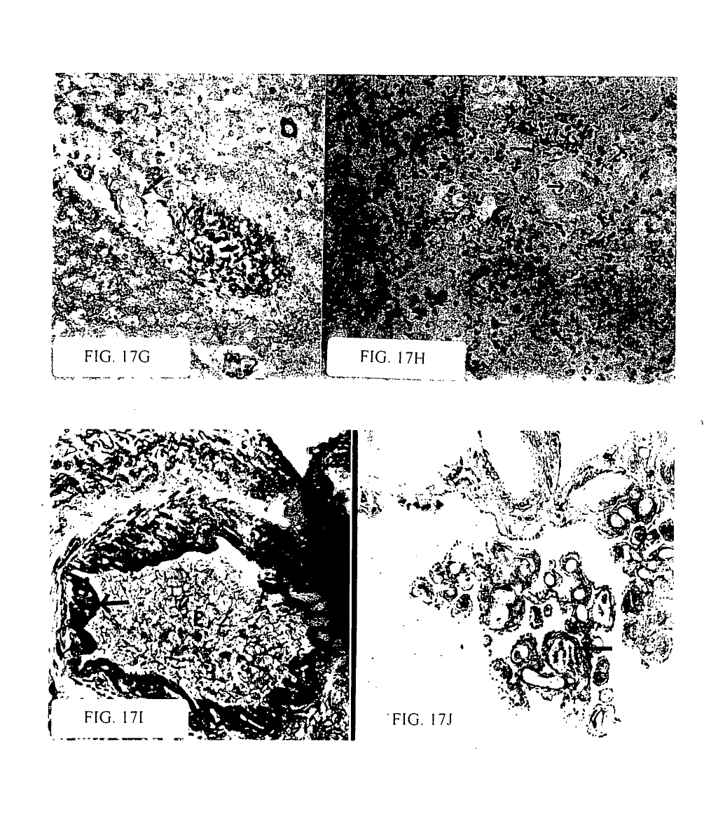

FIG. 17A. Choroid plexus. H&E stain Mag. ×40. The patient was a 77-year-old man with dementia, who died in a nursing home. Amyloid and amyloid-like material (arrow) occlude most small vessels, estimated at 80%. Three small vessels with patent lumen are labeled N. Black areas are calcified materials, due to MJA.

FIG. 17B. H&E stain Mag. ×400. Small vessels of choroid plexus, a vessel with a thin, normal appearing wall is labeled with an N in the lumen. Two adjacent vessels are also patent. The majority of remaining vessels is blocked (arrow) by amyloid appearing material. This patient was diagnosed clinically and pathologically as Alzheimer's disease. Steiner staining of these vessels shows heavy infection by MJA.

FIG. 17C. Choroid Plexus from lateral ventricle. Bielschowsky stain. Mag. ×400. The black stained material in the lumen of the vessels is an amorphous material resulting from MJA infection.

FIG. 17D. Cerebral Cortex. Bielschowsky stain. Mag. ×400. Neurons are laden with granules stained black, typical of that seen in Alzheimer's disease (arrow). Other scattered black structures are small vessels cut at various angles, also blocked by material as in larger vessels in Fig C.

FIG. 17E. Bielschowsky stain. Mag. ×400. Brain Cortex. Small vessels are filled with material stained black with this stain. This is confirming that these small vessels become occluded. The vessel wall is thin (arrow).

FIG. 17F. Steiner stain. Mag. ×1000. An arterial lumen is partly blocked by material not stained by this stain (large arrow). The empty space around the vessel is an artifact of preparation. The vessel wall shows areas of organism (black) and a dense ring of microorganism surrounds the area. Clumps of MJA are dispersed in surrounding brain, and the entire background contains large numbers of MJA. A neuron (small arrow) is stained black due to the microorganism. The Steiner stain shows that MJA is implicated in the patient's Alzheimer's disease.

FIG. 17G. Cerebral Cortex. Steiner stain. Mag. ×400. A blood vessel extending into the cortical tissue is occluded (arrow) by amyloid appearing material. A mass of MJA along the vessel track stains black with this stain; numerous microorganisms are also in the brain substance.

FIG. 17H. Cerebral Cortex. Steiner stain. Mag. ×1000. MJA is stained black and disbursed throughout the slide. The round and oval, pale yellow, vacuolated structures are a common feature of MJA infection.

FIG. 17I. Choroid plexus vein. Steiner stain. Mag. ×1000. Dense concentration of microorganism MJA is seen in the intima and sub-intima (arrow). Granular staining of the adventitia is due to small forms of MJA. Muscle cells in the vein wall are variably involved from a brown granular appearance through a solid black granular appearance. Erythrocytes in the lumen E show the microorganism attached to and within erythrocytes.

FIG. 17J. Choroid plexus. Steiner stain. Mag. ×100. The very heavy infection typical of MJA is seen in many vessels (arrow) where adventitia and muscle wall stain solidly black and even the lumen is filled with microorganism. Other vessels are affected to a variable degree but all vessels are involved.

### MJA in the Colon:

Patient Histories:

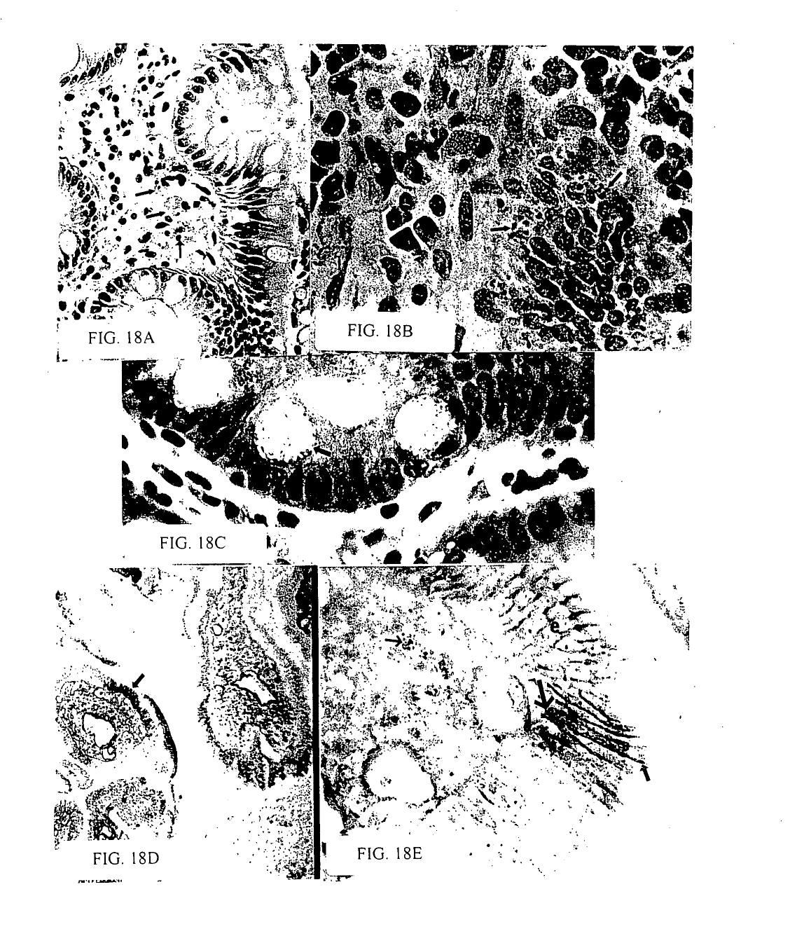

FIGS. 18A and B, 40-year old male, colon biopsy due to bloody stool.

Diabetes mellitus type 2

Hypertension

Obesity

Kidney failure

Elevated prostate specific antigen 790.93

FIG. 18C, 57-year old male, antral biopsy of the stomach.

Osteoarthritis of Knee

Hypercholesterolemia

Impotence

BPH (enlarged prostate)

GERD

Psoriasis

History of peptic ulcer

Allergic rhinitis

Abnormalities of hair

Hypertension

Seborrheic keratosis

Chronic antral gastritis with intestinal metaplasia

FIGS. 18D and E, female with history of Crohn's Disease and upper abdominal pain. Biopsy of duodenum.

FIG. 18A. Colonic mucosa. H&E stain. Mag. ×1000. The patient was a 40-year-old man with hematochezia and anemia. Other illnesses were diabetes mellitus, renal failure, hypertension, obesity and chronic prostatitis. The small round bodies in the lamina propria, also seen in round cytoplasmic bodies (arrows) indicate infection with MJA. The larger forms are clusters of microorganisms or phagolysosomes containing MJA.

FIG. 18B. Same patient as 18A. Colonic mucosa. H&E stain. Mag ×1000. The round bodies around the crypt of a colonic gland are forms of MJA. When these are seen on H&E stains they indicate infection with MJA. Figs. A & B demonstrate some of the variation in forms of MJA. Some bodies are in the space outside the gland. Mononuclear cells in the stroma are highly vacuolated with amber inclusions. These may represent parasitophorous vacuoles. Numerous plasma cells are present.

FIG. 18C. Gastric antrum. H&E stain. Mag. ×1000. The patient is a 57-year old male with gastrointestinal esophageal reflux disease (GERD), allergic rhinitis, psoriasis, hypertension and prostatitis. The antrum showed chronic gastritis with intestinal metaplasia. The metaplastic cells contain membrane bound parasitophorous vacuoles (arrow) containing MJA.

FIG. 18D. Biopsy of duodenum. Steiner stain. Mag. ×100. The villi are expanded due to congestion and inflammatory cells. Stromal cells and enterocytes contain microorganisms. The arrow indicates a patch of enterocytes with dense infection of microorganisms. Vessel walls are blackened due to numerous microorganisms. The heavy infection and multi-focal nature are typical of MJA infection.

FIG. 18E. Steiner stain. Mag. ×1000. Same patient as 18D. A patch of dense infection of MJA (large arrow) in the enterocyte (e) layer. The black streaks are due to microorganism in the intercellular space, a feature typical of MJA (middle size arrow) and a vessel is outlined by the microorganism in the wall. Cells in the lamina propria also contain microorganism (smallest arrow).

Any part of the digestive system may be involved by MJA, including the salivary glands.

### MJA in the Blood and White Blood Cells

Patient history:

A 69 year old male presented with marked weakness and shortness of breath. During performance of a fine needle aspiration the patient would fall asleep between maneuvers. He presented with an enlarged right scalene lymph node and a chest x-ray that showed enlarged mediastinal nodes. A fine needle aspiration of the scalene node revealed cells from a malignant lymphoma, lymphocytic type. A summary of his past illnesses is given below. Twelve years previously, a diagnosis of lymphocytic leukemia was made. The white blood cell count was 44,700 per cubic mm, with 35% lymphocytes. The patient was given conventional treatment. A fine needle aspiration of a scalene node in 2003 was diagnosed as malignant lymphoma, lymphocytic type. The patient's lymphocytes contained up to 10 phagosomes in each cell, which can represent thousands of MJA per cell.

Other illnesses of this patient are:

Acute maxillary sinusitis

Diabetes mellitus type 2

Peripheral neuropathy

Gastroenteritis

Benign hypertension

Senile cataract

Peripheral vascular disease

Selective immunoglobulin deficiency

Previous cholecystectomy (gall bladder removed)

Coronary artery disease

Benign prostatic hypertrophy

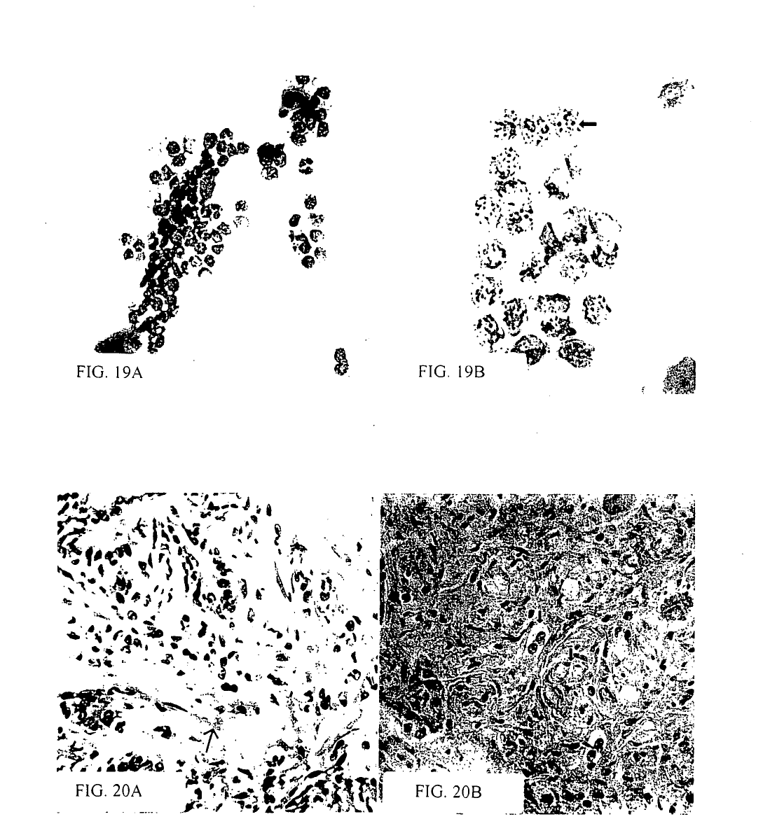

FIG. 19A. Diff-quick stain. Mag. ×400. Figure shows sheets of lymphocytes all in a similar stage of development. Most of the cells in Fig. A contain some organisms but a few appear void.

FIG. 19B. Same slide as Fig. A. Mag. ×1000. Detail in these cells shows most to contain numerous round and oval bodies, which are phagosomes each containing up to about 1,000 MJA.

### MJA in the Prostate Gland

FIG. 20A. Prostate Gland. H & E stain. Mag. ×1000. The prostate gland from a patient with enlarged prostate gland and chronic symptoms of prostatitis. Routine cultures were negative for any growth. The stromal and muscle cells are swollen showing ground glass appearance of muscle cells and fibrosis of stromal cells. There are no acute inflammatory cells although the glands are reduced in number.

FIG. 20B shows cellular reaction to MJA by muscle cells in the enlarged gland, causing the enlargement.

FIG. 20C shows MJA in the diseased gland in association with the disease, and therefore shows correlation between MJA and the diseased gland.

### MJA in the Breast

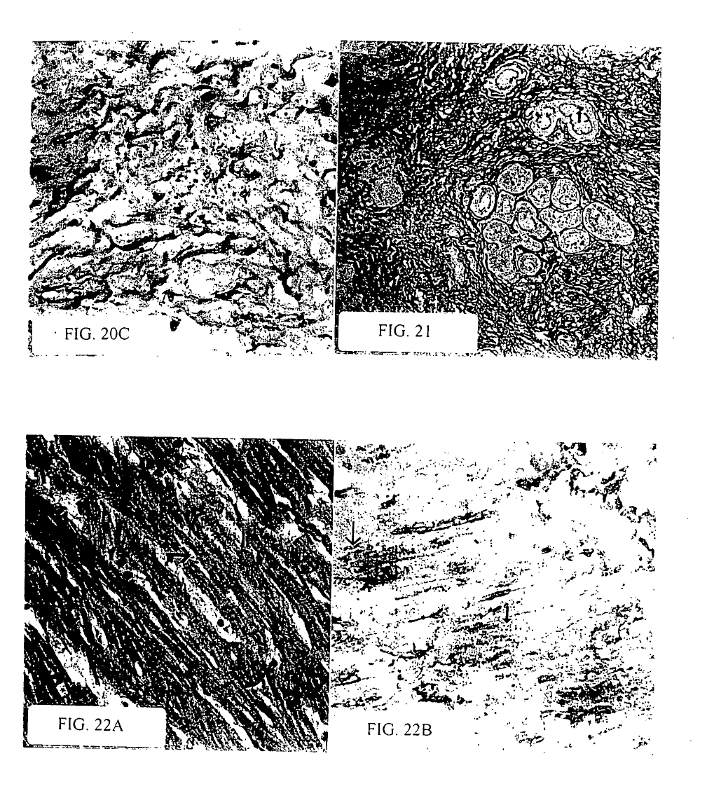

FIG. 21. Steiner stain, mag. ×100. Biopsy of a lump in the left breast from a 47-year-old female. There was a mass on mammography. The long arrow points to a mammary duct. Short arrow points to clump of microorganism stained black. There is loss of mammary lobules with persistence of small mammary ducts. These are set in a disordered fibrous stroma that includes repair and collapse. MJA is present in duct epithelium and in the stroma. In the usual course of the disease, the susceptible lobular epithelium is killed and disappears in time, leaving only the more resistant duct epithelium. Here, it appears that MJA is implicated in such loss.

### MJA in the Myocardium

FIG. 22A. H & E stain. Mag. ×400. The empty spaces (e.g., arrow), represent areas from which MJA has washed out during processing and is not stained by this stain. The presence of MJA in such samples can be shown by other means of staining, such as frozen section, to contain MJA. This sample shows pathology similar to that seen in congestive heart failure and high blood pressure.

FIG. 22B. Same biopsy as FIG. 22A. Steiner stain. Mag. ×1000. MJA, not well seen in the H & E stain in FIG. 22A, is clearly seen here, as black granules.

MJA in the Lung

FIGS. 23A and 23B. H & E stain. Mag. ×1000. Bronchoscopic biopsy of bronchial epithelium from a male patient with squamous cell carcinoma of the lung. Cells scraped off the bronchial surface show a high concentration of MJA within and outside of cells. This is exemplary of the high concentration MJA associated with lung and other cancers. For example, MJA has also been found associated with squamous cell carcinoma of the tonsil region (the salivary glands had been destroyed by MJA, adenocarcinoma of the endometrium, adenocarcinoma of the colon, oligodendroglioma of the brain, and undifferentiated small cell carcinoma of the lung.

### MJA in the Arteries

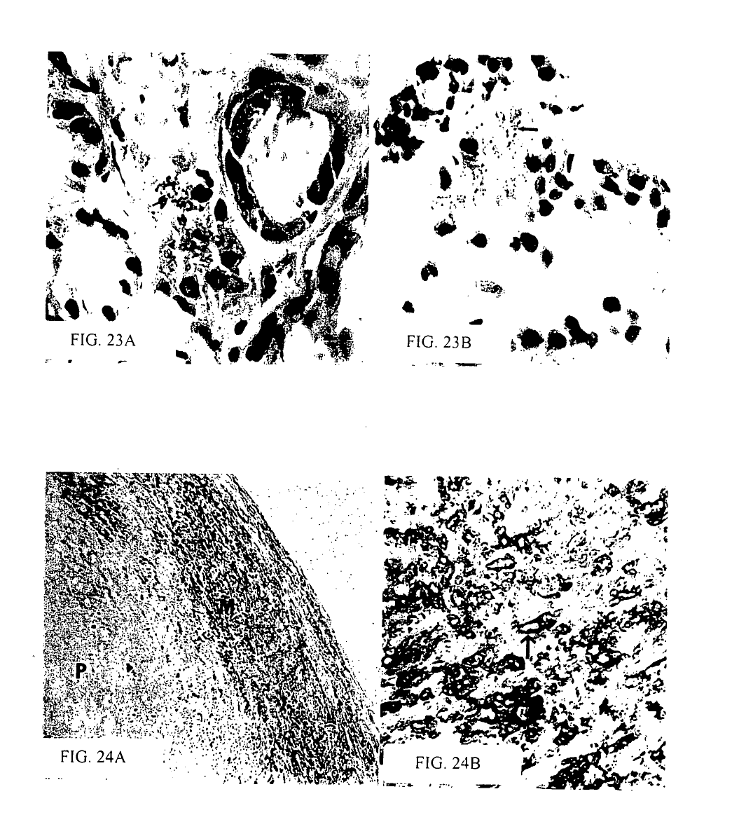

FIG. 24A. Carotid artery. Steiner stain. Mag. ×40. An occlusive lesion of the carotid artery of a man having difficulty with speech and movements of his right arm. Patient underwent removal of the occlusive lesion. The muscle M shows a high concentration of MJA showing as small black dots. The plaque portion (P) also has MJA but in lesser numbers.

FIG. 24B. Same slide as A. Mag. ×400. MJA is shown stained black.

### Some Further Election Micrographs

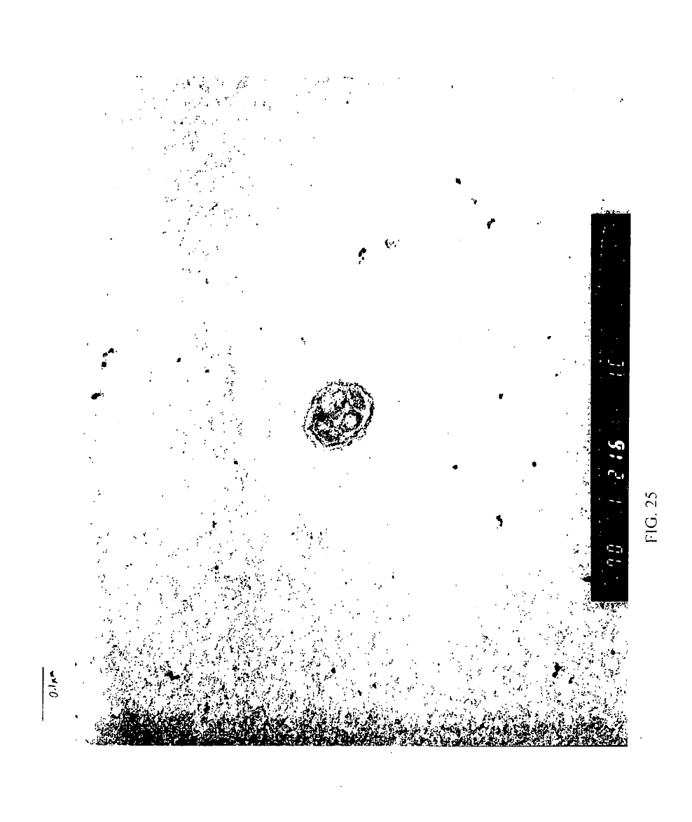

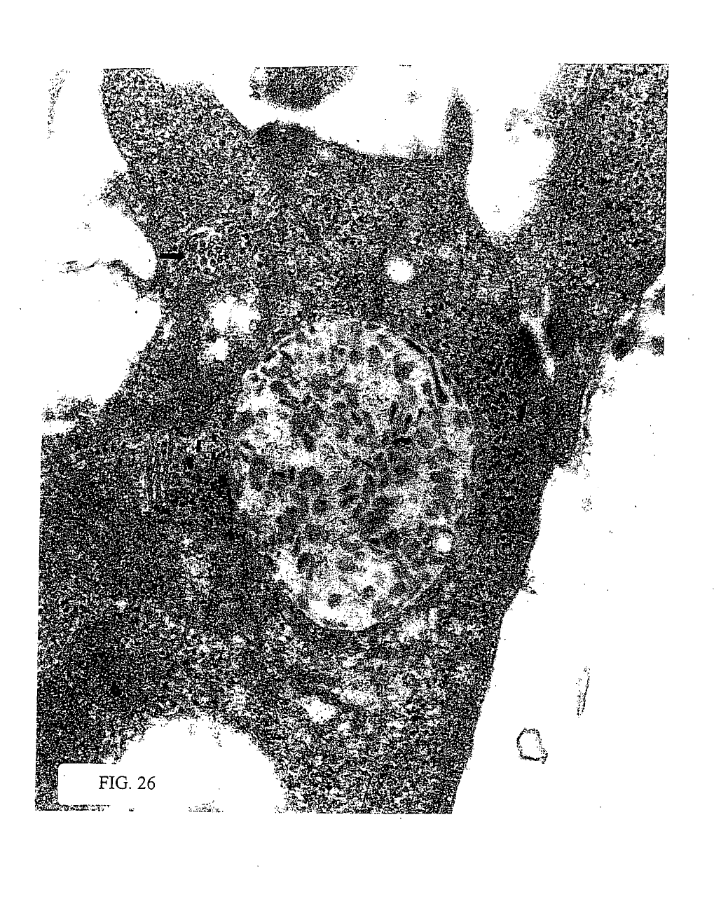

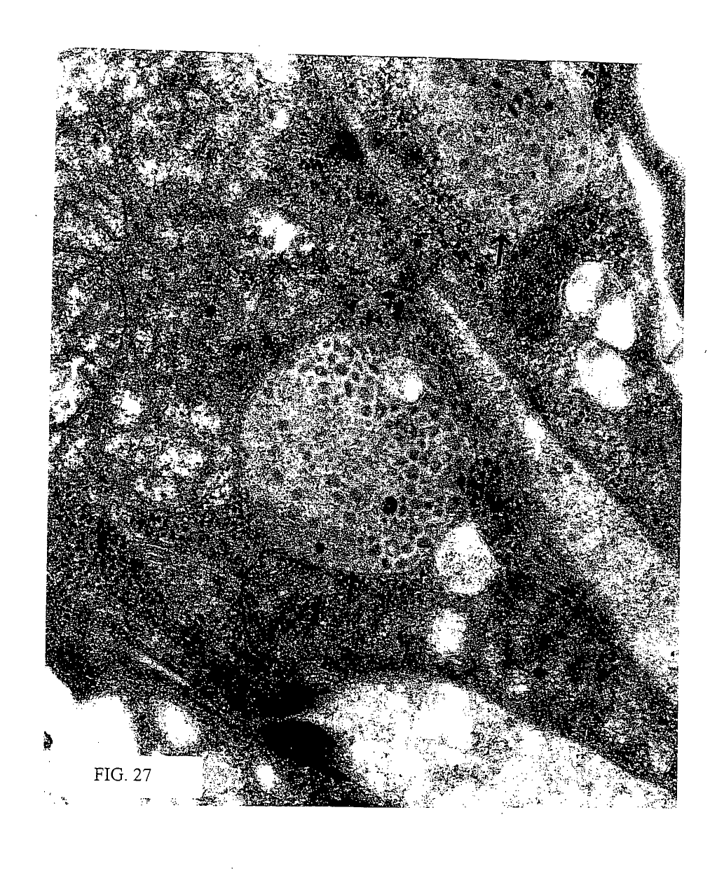

FIG. 25 is an electron micrograph of an erythrocyte from a patient with ulcerative colitis. A double membrane form of MJA is shown. The sample is from a drop of whole blood from a 40 year old male that was fixed in glutaraldehyde.

FIG. 26 is an electron micrograph, mag. ×88000, of an enterocyte from a patient with colitis. The open arrow points to a phagosome, a membrane lined sac, filled with MJA. The MJAs have double membranes and there is some variation in size of individual MJAs.

FIG. 27 is an electron micrograph, mag. ×88000, from the same patient as FIG. 26. Two phagosomes are shown filled with MJA.

FIG. 28 is an electron micrograph from the same patient as FIG. 26, mag. ×140000. The photo shows a phagosome containing MJA (arrow). The MJA have a double membranes.

The above comments and other perceptions of the inventor provide the following observations about MJA:

a. MJA may persist throughout the life of an individual, apparently causing periodic exacerbations of disease. Generally increasing damage is seen with increasing age. Initial infection can occur early in life and may possibly be present at birth.

b. Evidence of MJA is found in a large percentage of sick patients.

c. MJA is pleomorphic and has been found in spherical, elongated, pointed, and occasionally C-shaped forms. Various shapes of MJA have also been found when MJA has been grown in culture.

d. MJAs size is typically about 0.5 to 2.5 microns.

e. MJA has been seen with the following stains, a) Steiner stain, b) crystal violet stain, c) Genta stain, d) Gomori silver methenamine stain counter stained with hematoxylin, e) hematoxylin except inside RBCs, and f) Giemsa stain except inside RBCs. MJA has not been seen with the following stains, a) Wright stain as normally used in RBCs, b) Gram stain, c) eosin except as noted elsewhere herein, and d) acid fast stains.

f. MJA has been seen in electron micrographs. (See, e.g., FIGS. 1 and 25-28.)

g. MJA has been grown in culture in Vitacell from ATCC, RPMI-1640 Medium, modified, ATCC Number: 30-2001, with and without 20% fetal calf serum, and in chick embryo cells, and in the allantoic fluid and other portions of fertilized hen's eggs.

h. MJA has been found blocking small blood vessels, which blockage is fluorescent when stained by Eosin Y, appears to produce ischemic changes with hemorrhage and ulcerations of small and large bowel, and conforms to the pathogenesis of ulcerative colitis and Crohn's disease.

i. MJA in some cases occurs in very large numbers with little or no inflammation, although in other cases it is sometimes found in association with inflammatory lesions.

j. In Crohn's disease and other inflammatory bowel disease (IBD), MJA appears to change absorptive cells of the small bowel into cells resembling goblet cells. The sac-like structures in the cells, which resemble the sacs that are characteristic of real goblet cells, appear similar to parasitophorous vacuoles and contain MJA.

k. MJA binds to antibodies in a patient's serum when their own cells are incubated with the serum. Thus, MJA appears to be antigenic.

I. MJA is implicated in a wide variety of diseases including Alzheimer's disease, rheumatoid arthritis, ulcers, colitis, IBD, IBS, diabetes, atherosclerosis, hypertension, seborrheic keratosis, cancer.

Turning now to some other particular embodiments, the present invention comprises purified MJA (as exemplified by the deposit ATCC Accession No. PTA5003; MJA can be obtained from such deposit or from suitable natural sources, such as humans or other animals harboring MJA) as well as isolated and specifically identified MJA. The present invention also provides kits comprising a vessel containing MJA and a label specifically identifying the MJA. The vessel can be a vial, microscope slide or other suitable container. The MJA can be in an acellular culture medium, a cell culture medium, a tissue sample, or other suitable growth media. The kits can comprise one or more images of specifically identified MJA.

The present invention also comprises methods of obtaining MJA comprising: a) identifying an animal suspected of being infected with MJA; and, b) withdrawing a sample of tissue from the animal wherein the sample contains MJA. Similar methods can also serve to seek MJA in such an animal, for example a human patient, even where the sample does not include MJA. The methods can further comprise specifically identifying MJA from the tissue and, if desired, substantially purifying the MJA from the tissue and growing the MJA.

The present invention further comprises methods of specifically identifying MJA comprising: a) providing a sample of tissue suspected of containing MJA; b) examining the sample to determine the presence of MJA; and, c) specifically identifying MJA. The methods can further comprise, prior to the identifying, staining the sample with a stain suitable for MJA, and the examining can comprise magnifying the sample; other methods of detection, including indirect methods, can also be used. Where the methods comprise magnifying, the methods can also comprise creating a photomicrograph or other image of the MJA. The magnifying can be, for example, visible light microscopy, fluorescent light microscopy or electron microscopy (in which case the photomicrograph comprises an electronphotomicrograph), confocal microscopy or any other desired imaging modality. The image can be photographic, digital, videographic or otherwise as desired.

The present invention also provides kits comprising an image (hard copy or digital or other desired substrate) such as a photomicrograph and a label wherein the photomicrograph depicts at least one MJA and the label specifically identifies the MJA. The photomicrograph can be photographic paper and the label can be attached (e.g., glued or otherwise adhered) to the photomicrograph, the photomicrograph and the label can be digital representations that are digitally associated with each other, or combinations of the two or other imaging materials as desired. The photomicrograph can be a visible light photomicrograph, a fluorescent light photomicrograph, an electronphotomicrograph or other micrograph or imaging representation as desired.

In one aspect, the present invention comprises substantially purified MJA. In some embodiments, the MJA (ATCC as Accession No. PTA5003) can be isolated and specifically identified. The present invention also includes kits comprising a vessel containing isolated or substantially purified MJA and a label specifically identifying the MJA. The vessel can be a vial, microscope slide or other desired container, and the MJA can be in a cellular or acellular culture medium, a tissue sample or other desired growth, maintenance or holding substrate.

The present invention also comprises methods of obtaining MJA comprising: a) identifying an animal suspected of being infected with MJA; and, b) withdrawing a sample of tissue from the animal wherein the sample contains MJA. The methods can further comprise specifically identifying MJA from the tissue, substantially purifying the MJA from the tissue and, if desired, growing the MJA in a culture medium or other growth substrate.

Other methods provide for specifically identifying MJA. Such methods can comprise a) providing a sample of tissue suspected of containing MJA; b) examining the sample to determine the presence of MJA; and, c) specifically identifying MJA. Such examining of the sample can include comparing the MJA to the sample of MJA deposited with the ATCC as Accession No. PTA5003 or referring to one or more of the features discussed herein. Such methods can further comprise, prior to the identifying, staining the sample with a stain suitable for MJA, and the examining can comprise magnifying the MJA and creating an image of the MJA. Such magnifying can comprise, for example, visible light microscopy, fluorescent light microscopy or electron microscopy, and the image can comprise a photomicrograph such as an electronphotomicrograph, a visible light photomicrograph or a fluorescence photomicrograph.

Turning now to some other information and aspects, the discussion herein includes anti-MJA antibodies, anti-MJA screens and diagnostics, and anti-MJA therapeutics or drugs. The following sections include headers for convenience.

### 1. Anti-MJA Antibodies and other Anti-MJA Binding Partners

### Antibodies Generated Against MJA

Antibodies against MJA are generated using MJA itself as the antigen, although if desired isolated MJA proteins or peptides can be used, as can antigenic peptides derived from the amino acid sequence of MJA, then using desired antibody generation techniques such as those discussed herein. The specification will now discuss a variety of antibody types, methods, uses, etc., related to MJA. Preferably, the anti-MJA antibodies of the invention are specific substantially only for MJA, and have a high affinity for MJA. For example, a desired affinity is Kd=10⁻⁸M or better, for example 10⁻⁹M, 10⁻¹⁰M or 10⁻¹¹M.

### Antibodies Generally

In some embodiments, the present invention provides antibodies or other binding partners specific for MJA. Compositions and uses for such antibodies are contemplated, including diagnostic, medicament, and therapeutic uses. Various diagnostic, medicament, and therapeutic uses for other antibodies have been reviewed, for example, in Goldenberg et al., Semin. Cancer Biol., 1(3):217-225 (1990); Beck et al., Semin. Cancer Biol., 1(3):181-188 (1990); Niman, Immunol. Ser., 53:189-204 (1990); Endo, Nippon Igaku Hoshasen Gakkai Zasshi (Japan), 50(8):901-909 (1990); U.S. Pat. No. 6,214,984; and, U.S. Pat. No. 6,406,863.

Recognized immunoglobulin genes include the kappa, lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively.

An exemplary immunoglobulin (antibody) structural unit comprises a tetramer held together to make a Y-shape unit. In IgG, for example, each unit is composed of two identical pairs of polypeptide chains, each pair having one light (L) chain (about 25 kD) and one heavy (H) chain (about 50-70 kD), forming a general formula H₂ L₂ and a total molecular weight of about 150-200 kDa.

Typically, an antibody can be proteolytically cleaved by the proteinase papain into two identical Fab (fragment antigen binding) fragments and one Fc (fragment crystallizable) fragment. Each Fab fragment contains one binding site for the antigen, and the Fc portion mediates other aspects of the immune response. Each L chain is attached to one H chain by a disulfide bond. The two H chains are also attached to each other by disulfide bonds. Papain cleaves N-terminal to the disulfide bonds that hold the H chains together. Each of the resulting Fabs consists of an entire L chain plus the N-terminal half of an H chain; the Fc is composed of the C-terminal halves of two H chains.

Pepsin cleaves at numerous sites C-terminal to the inter-H disulfide bonds, resulting in the formation of a divalent fragment [F(ab′)] and many small fragments of the Fc portion. IgG heavy chains contain one N-terminal variable (V_{H}) plus three C-terminal constant (C_{H} 1, C_{H} 2 and C_{H} 3) regions. Light chains contain one N-terminal variable (V_{L}) and one C-terminal constant (C_{L}) region each. The N-terminus of each chain defines a variable region of about 100 to 110 or more amino acids primarily responsible for antigen recognition. Fabs consist of one V_{L}, V_{H}, C_{H} 1, and C_{L} region each. The V_{L} and V_{H} portions contain hypervariable segments (complementarity-determining regions or CDRs) that form the antibody combining site. The variable portion can also be referred to as a variable fragment (Fv).

The V_{L} and V_{H} portions of an antibody can be linked by a synthetic linker to form a single chain protein (scFv) which retains the same specificity and affinity for the antigen as the antibody itself. Bird, R. E., et al. (1988) “Single-chain antigen-binding proteins,” Science 242:423-426. A typical scFv can be a recombinant polypeptide composed of a V_{L} tethered to a V_{H} by a designed peptide, such as (Gly₄-Ser)₃, that links the carboxyl terminus of the V_{L} to the amino terminus of the V_{H} sequence. The construction of the DNA sequence encoding a scFv can be achieved by using a universal primer encoding the (Gly₄-Ser)₃ linker by polymerase chain reactions (PCR). Lake, D. F., et al. (1995) “Generation of diverse single-chain proteins using a universal (Gly₄-Ser)₃ encoding oligonucleotide,” Biotechniques 19:700-702.

### Anti-Idiotypic Antibodies

The present invention also encompasses anti-idiotypic antibodies, polyclonal, monoclonal, and otherwise, that are produced using the antibodies discussed herein as antigens. These antibodies are useful because they may mimic the structures of the MJA and can be used to bind anti-MJA specific antibodies.

Exemplary techniques for producing antibodies, including antibody fragments, include the following.

a. Antibody Preparation Methods

(iii) Human And Humanized Antibodies

### Human AB Generally

Human antibodies can be produced according to any desired method. For example, a humanized or fully human MJA antibody can be isolated using a yeast system. A desired MJA cDNA, or a truncated version if desired provided the MJA-specific antigenicity is not destroyed, can be used to express MJA epitopes in a yeast or other expression system, to screen a human antibody library. Positive clones will be further investigated for their specificity and efficiency. Desired clones can be maturated into full size antibodies with the Fc fragment. The ability of identified antibodies to identify MJA in samples or to inhibit the activities of MJA in vivo can be tested using, for example, the screening assays discussed herein or other screening modalities as desired. See, e.g., U.S. Pat. No. 6,406,863. High specificity and affinity are typically maintained. For example, a desired affinity is Kd=10⁻⁸ M or better, for example 10⁻⁹M, 10⁻¹⁰M or 10¹¹M.

In one embodiment, cDNA libraries of human heavy and light chain variable regions (V_{H} and V_{L}) are transferred into a yeast expression vector by direct homologous recombination between the sequences encoding the V_{H} and V_{L} regions and the yeast expression vector (which contains homologous recombination sites). The resulting expression vectors, which can be in plasmid form, are called scFv expression vectors. Each of the scFv expression vectors comprises a first nucleotide sequence encoding a human V_{H} region, a second nucleotide sequence encoding a human V_{L} region, and a linker sequence encoding a linker peptide that links the V_{H} and V_{L} regions together. The V_{H} region, the V_{L} region, and the linker peptide are expressed as a single fusion protein. This primary antibody library may reach a diversity generally between about 10⁶-10¹², more typically between about 10⁷-10¹², and preferably between about 10⁸-10¹².

Such scFv expression vectors are then co-transformed or mated along with expression vectors carrying MJA genes (or otherwise combined with expressible MJA) into host yeast cells.

Screening for antibody-MJA interaction may be conveniently carried out in yeast using a yeast two-hybrid method. For example, the library of human scFv expression vectors can be introduced into yeast cells. Expression of the human scFv antibody library in the yeast cells produces a library of human scFv fusion proteins, each fusion protein comprising a human scFv and an activation domain (AD) of a transcription activator. The yeast cells are also modified to express a recombinant fusion protein comprising a DNA-binding domain (BD) of the transcription activator and MJA. The yeast cells are further modified to express a reporter gene whose expression is under the control of a specific DNA binding site.

Upon binding of a human scFv antibody from the library to the MJA, the AD is brought into close proximity of BD, thereby causing transcriptional activation of a reporter gene downstream from a specific DNA binding site to which the BD binds. The library of human scFv expression vectors may contain the BD domain while the modified yeast cells express a fusion protein comprising the AD domain and the target antigen.

The yeast clones containing scFv antibodies with binding affinity to the MJA, also known as antibody leads, are selected based on phenotypes of the cells or other selectable markers. The plasmids encoding these primary antibody leads can be isolated and further characterized.

The sequences encoding V_{H} and V_{L} of the primary antibody leads can then be mutagenized in vitro to produce a secondary antibody library. The V_{H} and V_{L} sequences can be randomly mutagenized by “poison” PCR (or error-prone PCR), by DNA shuffling, or by any other way of random or site-directed mutagenesis (or cassette mutagenesis). After mutagenesis in the regions of V_{H} and V_{L}, the complexity of the secondary antibody library may reach 104 or more. Overall, the combined diversity or complexity of the total antibody libraries generated by using the methods of the present invention, including the primary and the secondary antibody libraries, may reach 1018 or more. The secondary antibody library is further screened for antibodies that bind MJA at high affinity by using the yeast-2-hybrid method as discussed above or other methods of screening in vivo or in vitro.

### Humanized AB Genarally

Methods for humanizing non-human antibodies are known and have been discussed in part above. Generally, a humanized antibody has one or more amino acid residues introduced into it from a source which is non-human. These non-human amino acid residues are often referred to as “import” residues, which are typically taken from an “import” variable domain. Humanization can be performed, for example, following the method of Winter and co-workers, Jones et al., Nature, 321:522-525 (1986); Riechmann et al., Nature, 332:323-327 (1988); Verhoeyen et al., Science, 239:1534-1536 (1988), by substituting rodent CDRs or CDR sequences for the corresponding sequences of a human antibody or otherwise as desired. Accordingly, such humanized antibodies are chimeric antibodies, U.S. Pat. No. 4,816,567, wherein substantially less than an intact human variable domain has been substituted by the corresponding sequence from a non-human species. Humanized antibodies are typically human antibodies in which some CDR residues and possibly some FR residues or other residues are substituted by residues from analogous sites in rodent antibodies.

The choice of human variable domains, both light and heavy, to be used in making humanized antibodies helps to reduce antigenicity. According to the so-called “best-fit” method, the sequence of the variable domain of a rodent antibody is screened against the entire library of known human variable-domain sequences. The human sequence that is closest to that of the rodent is then accepted as the human framework (FR) for the humanized antibody. Sims et al., J. Immunol., 151:2296 (1993); Chothia and Lesk, J. Mol. Biol., 196:901 (1987). Another method uses a particular framework derived from the consensus sequence of all human antibodies of a particular subgroup of light or heavy chains. The same framework may be used for several different humanized antibodies. Carter et al., Proc. Natl. Acad. Sci. USA, 89:4285 (1992); Presta et al., J. Immunol., 151:2623 (1993).

It is typically desirable that antibodies be humanized with retention of high affinity for MJA and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available. Computer programs are available that illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind MJA. In this way, FR residues can be selected and combined from the consensus and import sequences so that the desired antibody characteristic, such as increased affinity for MJA, is achieved. In general, CDR residues are directly and most substantially involved in influencing antigen binding.

It is also possible to produce transgenic animals (e.g., mice) that are capable, upon immunization, of producing a full repertoire of human antibodies in the absence of endogenous immunoglobulin production. For example, the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA. 90:2551-255 (1993); Jakobovits et al., Nature, 362:255-258 (1993); Bruggemann et al., Year Immuno., 7:33 (1993). Human antibodies can also be produced in phage-display libraries, Hoogenboom and Winter, J. Mol. Biol., 227:381 (1991); Marks et al., J. Mol. Biol., 222:581 (1991).

(ii) Polyclonal Antibodies

### Antibody Prep—Polyclonal:

Polyclonal antibodies are generally raised in animals by multiple subcutaneous (sc) or intraperitoneal (ip) injections of MJA or an antigenic sub-part thereof, and an adjuvant. It may be useful to conjugate the MJA to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

### Antibody Prep-Adjuvants (Can Be Useful for All ABS)

Suitable adjuvants for the vaccination of animals for the production of polyclonal, monoclonal, and other antibodies include but are not limited to Adjuvant 65 (containing peanut oil, mannide monooleate, and aluminum monostearate); Freund's complete or incomplete adjuvant; mineral gels such as aluminum hydroxide, aluminum phosphate, and alum; surfactants such as hexadecylamine, octadecylamine, lysolecithin, dimethyldioctadecylammonium bromide, N,N-dioctadecyl-N′,N′-bis(2-hydroxymethyl) propanediamine, methoxyhexadecylglycerol, and pluronic polyols; polyanions such as pyran, dextran sulfate, poly IC, polyacrylic acid, and carbopol; peptides such as muramyl dipeptide, dimethylglycine, tuftsin, stress proteins, core-containing proteins from a positive stranded RNA virus, see U.S. Pat. No. 6,153,378; and, oil emulsions. The polypeptides could also be administered following incorporation into liposomes or other microcarriers.

Information concerning adjuvants and various aspects of immunoassays are discussed, e.g., in the series by P. Tijssen, Practice and Theory of Enzyme Immunoassays, 3rd Edition (1987), Elsevier, New York. Other useful references covering methods for preparing polyclonal antisera include Microbiology, Hoeber Medical Division, Harper and Row (1969); Landsteiner, Specificity of Serological Reactions, Dover Publications, New York (1962); and, Williams, et al., Methods in Immunology and Immunochemistry, Vol. 1, Academic Press, New York (1967).

Animals can be immunized against the MJA, immunogenic conjugates, or derivatives by combining 1 mg or 1 μg of the MJA or conjugate (e.g., for rabbits or mice, respectively) with 3 volumes of Freund's complete adjuvant and injecting the solution intradermally at multiple sites. One month later the animals are boosted with ⅕ to 1/10 the original amount of MJA or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the MJA, but conjugated to a different protein or through a different cross-linking reagent. Conjugates also can be made in recombinant cell culture as protein fusions. In addition, aggregating agents such as alum can be suitably used to enhance the immune response.

(iii) Monoclonal Antibodies

### Antibody Prep—Monoclonal

Monoclonal antibodies are obtained from a population of substantially homogeneous antibodies, e.g., the individual antibodies comprising the population are identical except for possible naturally occurring mutations that may be present in minor amounts. For example, monoclonal antibodies can be made using the hybridoma method first discussed by Kohler and Milstein, Nature, 256:495 (1975), or can be made by recombinant DNA methods.

In the hybridoma method, a mouse, or other appropriate host animal, such as a hamster, is immunized as discussed above to elicit lymphocytes that produce or are capable of producing antibodies that will bind specifically to the protein used for immunization. Alternatively, lymphocytes may be immunized in vitro. Lymphocytes then are fused with myeloma cells using a suitable fusing agent, such as polyethylene glycol, to form a hybridoma cell. Goding, Monoclonal Antibodies: Principles and Practice, pp. 59-103, Academic Press (1986).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium, for example murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA. Human myeloma and mouse-human heteromyeloma cell lines have also been discussed for the production of human monoclonal antibodies, Kozbor, J. Immunol., 133:3001 (1984); Brodeur et al., Monoclonal Antibody Production Techniques and Applications, pp. 51-63, Marcel Dekker, Inc., New York (1987).

Culture medium in which hybridoma cells are growing is assayed for production of monoclonal antibodies directed against MJA. The binding specificity of monoclonal antibodies produced by hybridoma cells can be determined by immunoprecipitation or by an in vitro binding assay, such as radioimmunoassay (RIA) or enzyme-linked immunosorbent assay (ELISA). The binding affinity of the monoclonal antibody can, for example, be determined by the Scatchard analysis of Munson and Pollard, Anal. Biochem., 107:220 (1980).

After hybridoma cells are identified that produce antibodies of the desired specificity, affinity, or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-SEPHAROSE™, hydroxyapatite chromatography, gel electrophoresis, dialysis, or affinity chromatography.

DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which can then be transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Review articles on recombinant expression in bacteria of DNA encoding antibody include Skerra et al., Curr. Opinion in Immunol., 5:256-262 (1993), and Pluckthun, Immunol. Revs., 130:151-188 (1992).

### MOABS—Combinatorial

In a further embodiment, antibodies or antibody fragments can be isolated from antibody phage libraries generated using the techniques discussed in McCafferty et al., Nature, 348:552-554 (1990), using MJA to select for a suitable antibody or antibody fragment. The discussion above about creating human antibodies in yeast is one example of an approach for making combinatorial antibodies. Clackson et al., Nature, 352:624-628 (1991) and Marks et al., J. Mol. Biol., 222:581-597 (1991) discuss the isolation of murine and human antibodies, respectively, using phage libraries. Subsequent publications discuss the production of high affinity (nM range) human antibodies by chain shuffling, Marks et al., Biotechnology, 10:779-783 (1992), as well as combinatorial infection and in vivo recombination as strategies for constructing very large phage libraries, Waterhouse et al., Nuc. Acids. Res., 21:2265-2266 (1993). Combinatorial antibodies are also discussed in Huse et al., Science 246:1275-1281 (1989), and Sastry et al., Proc. Natl. Acad. Sci. USA, 86:5728-5732 (1989), and Alting-Mees et al., Strategies in Molecular Biology 3:1-9 (1990). These references discuss a system commercially available from Stratacyte, La Jolla, Calif. USA. Briefly, mRNA is isolated from a B cell population and utilized to create heavy and light chain immunoglobulin cDNA expression libraries in the λIMMUNOZAP(H) and λIMMUNOZAP(L) vectors. These vectors may be screened individually or co-expressed to form Fab fragments or antibodies, see Huse et al., supra; see also Sastry et al., supra. Positive plaques can subsequently be converted to a non-lytic plasmid, which allows for high-level expression of monoclonal antibody fragments from E. coli.

### Humanied MOAB:

Binding partners can also be constructed utilizing recombinant DNA techniques to incorporate the variable regions of a gene that encode a specifically binding antibody. The construction of these binding partners can be readily accomplished in view of the present application. See Larrick et al., Biotechnology, 7:934-938 (1989); Riechmann et al., Nature, 332:323-327 (1988); Roberts et al., Nature, 328:731-734 (1987); Verhoeyen et al., Science 239:1534-1536 (1988); Chaudhary et al., Nature, 339:394-397 (1989); see also U.S. Pat. No. 5,132,405 entitled “Biosynthetic Antibody Binding Sites”.) For example, the DNA can be modified by substituting the coding sequence for human heavy- and light-chain constant domains in place of homologous murine sequences, U.S. Pat. No. 4,816,567; Morrison, et al., Proc. Nat. Acad. Sci., 81:6851 (1984), or by covalently joining to the immunoglobulin coding sequence all or part of the coding sequence for a non-immunoglobulin polypeptide. In another example, DNA segments encoding the desired antigen-binding domains specific for MJA are amplified from appropriate hybridomas and inserted directly into the genome of a cell that produces human antibodies. See Verhoeyen et al., supra; see also Reichmann et al., supra. Some of these techniques transfer the antigen-binding site of a specifically binding mouse or rat monoclonal antibody or the like to a human antibody. Such antibodies can be preferable for therapeutic use in humans because they are typically not as antigenic as rat or mouse antibodies.

In an alternative embodiment, genes that encode the variable region from a hybridoma producing a monoclonal antibody of interest can be amplified using oligonucleotide primers for the variable region. These primers may be synthesized, or may be purchased from commercially available sources. For instance, primers for mouse and human variable regions including, among others, primers for V_{H}a, V_{H}b, V_{H}c, V_{H}d, C_{H}1, V_{L}, and C_{L} regions are available from Stratacyte (La Jolla, Calif.). These primers may be utilized to amplify heavy- or light-chain variable regions, which may then be inserted into vectors such as IMMUNOZAP™(H) or IMMUNOZAP™(L) (Stratacyte), respectively. These vectors may then be introduced into E. coli for expression. Utilizing these techniques, large amounts of a single-chain protein containing a fusion of the V_{H} and V_{L} domains may be produced, see Bird et al., Science 242:423-426 (1988).

### Antibody Substitions—Non-Immunoglubulins Polypeptides (All ABS)

Non-immunoglobulin polypeptides can be substituted in monoclonal and other antibodies discussed herein for the constant domains of an antibody, or they can be substituted for the variable domains of one antigen-combining site of an antibody to create a chimeric bivalent antibody comprising one antigen-combining site having specificity for MJA and another antigen-combining site having specificity for a different antigen. Although not necessarily antibody substitutions, the antibodies herein can also be chemically conjugated to a detectable moiety or other desired moiety.

### Chimerics

Chimeric or hybrid antibodies can also be prepared in vitro using known methods in synthetic protein chemistry, including those involving crosslinking agents, in view of the present application. For example, immunotoxins may be constructed using a disulfide-exchange reaction or by forming a thioether bond. Examples of suitable reagents for this purpose include iminothiolate and methyl-4-mercaptobutyrimidate.

### Antibody Labeling (All ABS)

For diagnostic applications or otherwise as desired, and for monoclonal and other antibodies discussed herein, the antibodies and other binding partners typically can be labeled with a detectable moiety. The detectable moiety can be any moiety that is capable of producing, either directly or indirectly, a detectable signal. For example, the detectable moiety may be a radioisotope, such as ³H, ¹⁴C, ³²P, ³⁵S, or ¹²⁵I; a fluorescent or chemiluminescent compound, such as fluorescein isothiocyanate, rhodamine, or luciferin; or an enzyme, such as alkaline phosphatase, beta-galactosidase, or horseradish peroxidase, avidin, and biotin. Any desired method conjugating the antibody or binding partner to the detectable moiety may be employed, including those methods discussed by Hunter et al., Nature, 144:945 (1962); David et al., Biochemistry, 13:1014 (1974); Pain et al., J. Immunol. Meth., 40:219 (1981); and Nygren, J. Histochem. Cytochem., 30:407 (1982).

(iv) Antibody Fragments

### Antibody Fragments

Various techniques have been developed for the production of antibody fragments. Such fragments can be derived via proteolytic digestion of intact antibodies, see, e.g., Morimoto et al., J. Biochem. Biophys. Meth. 24:107-117 (1992) and Brennan et al., Science, 229:81 (1985). Fragments can also be produced directly by recombinant host cells. For example, antibody fragments can be isolated from antibody phage libraries discussed above. Fab′-SH fragments can be directly recovered from E. coli and chemically coupled to form F(ab′)₂ fragments, Carter et al., Biotechnology 10:163-167 (1992). F(ab′)₂ fragments can be isolated directly from recombinant host cell culture. Other techniques for the production of antibody fragments will be apparent to the skilled practitioner.

(v) Bispecific Antibodies

### Bispecific Antibidies Generally

Bispecific antibodies (BsAbs) are antibodies that have binding specificities for at least two different antigens. Bispecific antibodies can be derived from full-length antibodies or from antibody fragments, e.g., F(ab′)₂ bispecific antibodies.

Methods for making bispecific antibodies are known. Traditional production of full-length bispecific antibodies is based on the coexpression of two immunoglobulin heavy chain-light chain pairs, where the two chains have different specificities, Millstein and Cuello, Nature, 305:537-539 (1983). Because of the random assortment of immunoglobulin heavy and light chains, these hybridomas (quadromas) produce a mixture of potentially 10 different antibody molecules, of which only one has the correct bispecific structure. Purification of the correct molecule, which is usually accomplished by affinity chromatography steps, is rather cumbersome, and the product yields can be low. Similar procedures are discussed in WO 93/08829, and in Traunecker et al., E.M.B.O. J., 10:3655-3659(1991).

According to another approach, antibody variable domains containing the desired binding specificities (antibody-antigen combining sites) are fused to immunoglobulin constant domain sequences. The fusion is preferably with an immunoglobulin heavy chain constant domain, comprising at least part of the hinge, C_{H} 2, and C_{H} 3 regions. It is preferred to have the first heavy-chain constant region (C_{H} 1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

### Antibodies—Hybrid Immunnoglubin Heavy Chain

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure may facilitate the separation of the desired bispecific compound from unwanted immunoglobulin chain combinations, as the presence of an immunoglobulin light chain in only one half of the bispecific molecule provides for a facile method of separation. This approach is discussed in WO 94/04690. For further details of generating bispecific antibodies see, for example, Suresh et al., Meth. Enzymol., 121:210 (1986).

### Antibodies—Cross-Linked or “Heteroconjugate”

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells, U.S. Pat. No. 4,676,980), and for treatment of HIV infection, WO 91/00360, WO 92/200373, and EP 03089). Heteroconjugate antibodies may be made using any convenient cross-linking methods. Suitable cross-linking agents are well known, and are discussed in U.S. Pat. No. 4,676,980, along with a number of cross-linking techniques.

### Antibodies—Diabodies

The “diabody” technology discussed by Hollinger et al., Proc. Natl. Acad. Sci. USA, 90:6444-6448 (1993) has provided an alternative mechanism for making BsAb fragments. The fragments comprise a heavy-chain variable domain (V_{H}) connected to a light-chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites.

Another strategy for making BsAb fragments by the use of single-chain Fv (sFv) dimers has also been reported. See Gruber et al., J. Immunol., 152:5368 (1994). These researchers designed an antibody comprising the V_{H} and V_{L} domains of a first antibody joined by a 25-amino-acid-residue linker to the V_{H} and V_{L} domains of a second antibody. The refolded molecule bound to fluorescein and the T-cell receptor and redirected the lysis of human tumor cells that had fluorescein covalently linked to their surface.

b. Other Binding Partners

Techniques for generating other MJA-specific binding partners are known.

For example, bispecific antibodies from antibody fragments have also been discussed in the literature, and bispecific binding partners can be prepared using chemical linkage. Brennan et al., Science, 229:81 (1985) discuss a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the BsAb. The BsAbs produced can be used as agents for the selective immobilization of enzymes.

Fab′-SH fragments can be directly recovered from E. coli, which can be chemically coupled to form bispecific binding partners. Shalaby et al., J. Exp. Med., 175:217-225 (1992) discuss the production of a fully humanized BsAb F(ab′)₂ molecule. Each Fab′fragment was separately secreted from E. coli and subjected to directed chemical coupling in vitro to form the BsAb. The BsAb thus formed was able to bind to cells overexpressing the HER2 receptor and normal human T cells, as well as trigger the lytic activity of human cytotoxic lymphocytes against human breast tumor targets. See also Rodriguez, et al., Int. J. Cancers (Suppl.) 7:45-50 (1992).

Various techniques for making and isolating BsAb fragments directly from recombinant cell culture have also been discussed. For example, bispecific F(ab′)₂ heterodimers have been produced using leucine zippers. Kostelny, et al., J. Immunol., 148(5):1547-1553 (1992). The leucine zipper peptides from the Fos and Jun proteins were linked to the Fab′portions of two different antibodies by gene fusion. The antibody homodimers were reduced at the hinge region to form monomers and then re-oxidized to form the antibody heterodimers.

c. Antibody Purification

Anti-MJA antibodies and other proteins can be substantially purified by standard methods, including but not limited to salt or alcohol precipitation, preparative disc-gel electrophoresis, isoelectric focusing, high pressure liquid chromatography (HPLC), reversed-phase HPLC, gel filtration, cation and anion exchange, partition chromatography, and countercurrent distribution. Exemplary purification methods are discussed, e.g., in Guide to Protein Purification, Methods in Enzymology, Vol. 182, M. Deutscher, Ed., Academic Press, New York, N.Y. (1990). Purification steps can be followed as part of carrying out assays for ligand binding activity. Particularly where MJA is being isolated from a cellular or tissue source, it may be desirable to include one or more inhibitors of proteolytic enzymes in the assay system, such as phenylmethylsulfonyl fluoride (PMSF).

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992), discuss a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Antibodies can also be substantially purified, for example, using low pH hydrophobic interaction chromatography (LPHIC) techniques. U.S. Pat No. 6,214,984.

d. Identifying Anti-MJA Antibodies or Other MJA-Specific Binding Partners that Inhibit MJA

The present invention further provides systems and methods for identifying anti-MJA antibodies or other MJA-specific binding partners that bind to and antagonize MJA activity. Antagonist anti-MJA antibodies can be useful in treatment and management of a variety of diseases including Alzheimer's disease, rheumatoid arthritis, ulcers, colitis, IBD, IBS, diabetes, atherosclerosis, hypertension, seborrheic keratosis, cancer, or any other disease or disorder in which MJA is involved.

To evaluate the effect of a candidate anti-MJA antibody or other MJA-specific binding partner on MJA activity, a biological activity assay can be performed wherein the candidate anti-MJA antibody or other MJA-specific binding partner is added to an incubation mixture. Briefly the candidate anti-MJA antibody and MJA polypeptide are incubated under conditions sufficient to allow the components to interact. Subsequently, the effect of the candidate anti-MJA antibody on MJA biological activity is measured. The desired decrease in MJA biological activity can be measured by, for example, adding a radioactive compound such as ³² P-ATP to the mixture of components, and observing radioactive incorporation into a suitable substrate for the activity of MJA, such as the increased presence/absence of a MJA metabolite, to determine whether the candidate anti-MJA antibody inhibits, stimulates, or has no effect on MJA biological activity.

Within such assays, the candidate anti-MJA antibody or other MJA-specific binding partner may be preincubated with MJA before addition of substrate or the substrate may be preincubated with the candidate anti-MJA antibody before the addition of MJA. Any of these assays can further be modified by removing the candidate anti-MJA antibody after the initial preincubation step. In general, a suitable amount of candidate anti-MJA antibody or other MJA-specific binding partner for use in such an assay ranges from about 0.1 μM to about 10 μM. The effect of the agent on MJA biological activity may then be evaluated by quantifying the change in the amount or activity of the substrate, and comparing the level of biological activity with that achieved using MJA polypeptide without the addition of the candidate anti-MJA antibody.

In another approach, labeled MJA can be incubated with candidate anti-MJA antibodies or other binding partner. The amount of the label is then determined. Typically, a given amount of labeled MJA is contacted with increasing amounts of anti-MJA antibodies, and the amount of the bound labeled MJA is measured after removing unbound labeled material by washing. As the amount of the label is increased, a point is eventually reached at which all MJA binding sites are occupied or saturated. Specific MJA binding is abolished by a large excess of unlabeled MJA.

### 2. Systems and Methods for Screening for MJA Antagonist

a. Generally

### Screening for Antagonist

The invention provides for the discovery of antagonists of MJA, including selective antagonists of MJA, as discussed herein that can be useful in treatment and management of a variety of diseases implicated with NJA, including Alzheimer's disease, rheumatoid arthritis, ulcers, colitis, IBD, IBS, diabetes, atherosclerosis, hypertension, seborrheic keratosis, and cancer, or any other disease or disorder in which MJA is involved.

Thus, the biological activity or functionality of MJA can be employed in screening systems to identify antagonists of MJA. These systems provide methods for bringing together the MJA, an appropriate known metabolite, bioactivity, etc., and a sample to be tested for the presence of antagonism.

### Constitutively Active MJA for Screening for Antagonist

The use of a constitutively active MJA either occurring naturally without further modification or after appropriate point mutations, deletions, or the like, is one approach to screening for antagonists and of in vivo use of such antagonists to attribute to or clarify a role of MJA.

### Exemplary Function Assay for MJA Antagonist

Biological activities of, or mediated by, MJA can be monitored for antagonistic activities. Such parameters include but are not limited to metabolization of detectable (tagged, if desired) metabolites, changes in cell growth rate of the MJA or of a sample infected with MJA, activity/health of an organism infected with MJA, etc. Examples of such methods include measurement of the effects of a putative MJA antagonist on MJA-mediated biological activity or functionality of MJA compared to the biological activity or functionality without the putative MJA antagonist. Antagonists of MJA may also be identified directly by using functional assays. An antagonist may or may not directly inhibit or enhance the activity of MJA.

Activities of an antagonist on MJA may be also measured in cellular or organismal models for altered biological activity or functionality of MJA, including for effects on lung disorders including; neurological disorders including Alzheimer's disease; cancers including breast, colon, lung, and prostate; atherosclerosis; heart disease including congestive heart failure; gastrointestinal disorders including ulcerative colitis, IBD and Crohn's disease, and immune and autoimmune disorders such as allergic rhinitis and rheumatoid arthritis.

### 3. Pharmaceutal Compositions and Uses Related to Anti-MJA Antibodies and Other Anti-MJA Binding Partners

### Generally

Pharmaceutical compositions, and uses, etc., for anti-MJA antibiotics such as drugs and anti-MJA antibodies will now be discussed. As with other parts of this application, this section does not contain the entire discussion of therapeutic uses or compositions, etc., other sections discuss both antibodies and therapeutics, and the discussion in this section applies to certain other aspects discussed herein. As also noted elsewhere herein, antibiotics, drugs, antibodies and other agents having a desired effect on MJA activity are typically administered to a patient (either prophylactically or for treatment of an existing disease) to modulate the biological activity of MJA. A “patient” may be any animal, typically a mammal or farm animal, and may be human. The patient, or animal, may be afflicted with a disease or condition associated with an overabundance of or overactivity by MJA, or other undesirable effect of MJA, or may be free of detectable disease. Accordingly, the treatment may be of an existing disease or may be prophylactic. Treatments can also be for health or body enhancements not directly related to diseases or negative conditions, such as, if appropriate, improving muscle, brain or sensory function, for example in the treatment of Alzheimer's disease or rheumatoid arthritis.

Anti-MJA drugs can be administered in a pharmaceutical amount to reduce one or more of the symptoms associated with, for example, Alzheimer's disease, rheumatoid arthritis, ulcers, colitis, IBD, IBS, diabetes, atherosclerosis, hypertension, seborrheic keratosis, cancer, or any other disease or disorder in which MJA is involved.

Suitable anti-MJA drugs include antibiotics, anti-MJA antibodies and other MJA antagonists. Exemplary antibiotics anti-MJA drugs include doxycycline, tetracycline, quinolones such as norfloxacin, pefloxacin, ciprofloxacin, oflaxacin, ruvloxacin, Ro-091168, KB-5246, 4-quinolone, isothiazolo-quinolone, pyrridoquinolone and nalidixic acid, nitroimidazole, nitrofuran, rifamycin (rifampicin), streptomycin, aminocyclitol, chloramphenicol, kanamycin, gentamycin, erythromycin, azithromycin, lincomycin and clindamycin.

Pharmaceutical anti-MJA compositions may also be configured for use with secondary compounds, which may be non-anti-MJA compounds (i.e., inactive with respect to MJA but effective to treat or reduce at least one symptom of the given disease whether such symptom is specifically tied to MJA or caused by other agents) and can be biologically or therapeutically active or inactive. Such agents include an anti-Alzheimer's disease drug, anti-rheumatoid arthritis drug, anti-colitis drug, anti-IBD drug, anti-diabetes drug, anti-atherosclerosis drug, anti-hypertension drug, anti-seborrheic keratosis drug, and an anti-cancer drug, or a drug against any other disease or disorder in which MJA is involved.

Representative anti-disease drugs are well known for each of the diseases discussed herein. Nevertheless, for some of the diseases the following lists may be instructive.

Representative insulin-dependent diabetes agents include nateglinide, repaglinide, thiazolidinedione derivatives (glitazones, e.g., rosiglitazone, rosiglitazone and pioglitazone), sulfonyl urea derivatives, metformin, biguanide mefformin, various forms of human insulin such as those in Jens Brange, Galenics of Insulin, The Physico-chemical and Pharmaceutical Aspects of Insulin and Insulin Preparations (springer-Verlag, N.Y., 1987), page 17-40, which include regular insulin, NPH (Neutral Protamine Hagedorn)-insulin, also called Isophane Insulin, 70/30 insulin, composed of 70% NPH-insulin and 30% Regular insulin, Semilente insulin, UltraLente insulin, Lente insulin, and HUMALOG® insulin lispro injection (rDNA origin), and the appropriate drugs and agents in Diabetes Mellitus—Theory and Practice, fourth edition, Harold Rifkin, MD, Ed. (Elsevier, N.Y., 1990), Chapter 29, and U.S. Pharmacist, 18 (November Suppl.) p. 38-40 (1993)

Representative anti-atherosclerosis agents include, for example, C16-plus polyunsaturated fatty acid compounds comprising at least one pair of double bonds in a conjugated position (U.S. Pat. No. 6,555,579), 2-hydrazeno adenosines and other adenosine A₂ receptor agonists (U.S. Pat. No. 5,278,150), substituted acid derivatives as discussed in U.S. Pat. No. 6,414,002, lipoxygenase inhibitors, ACAT inhibitors, antioxidants, PPAR agonists, phospholipase inhibitors including PLA-2 inhibitors.

Representative anti-cancer agents include paclitaxel, docetaxel, alkylating agents including mechlorethamine, chlorambucil, cyclophosphamide, melphalan and ifosfarnide; antimetabolites including methotrexate, 6-mercaptopurine, 5-fluorouracil (e.g., Efudex®) and cytarabine; plant alkaloids including vinblastine, vincristine (e.g, Oncovin®) and etoposide; antibiotics including doxorubicin (Adriamycin®), daunomycin, bleomycin, and mitomycin; gemcitabine hydrochloride (Gemzar®); nitrosureas including carmustine and lomustine; inorganic ions including cisplatin (Platinol-AQ®); biological response modifiers including interferon; enzymes including asparaginase; and hormones including estrogen, tamoxifen and flutamide, including their homologs, analogs, fragments, derivatives, pharmaceutical salts and mixtures thereof.

Such additional agents can be included a single composition with the anti-MJA antibody or in a second (or more) additional composition. Thus, are additional appetite activator (or other additional agent) can be administered before the anti-MJA composition, simultaneously with the anti-MJA composition, or after the anti-MJA composition. For example, the anti-MJA composition can be administered intravenously while the second agent is administered orally.

### Pharmaceutal Formulations

Therapeutic formulations of the anti-MJA composition can be configured by mixing the anti-MJA antibiotic having a desired degree of purity with at least one optional pharmaceutically acceptable carrier, adjuvant, excipient, buffer and diluent (Remington's Pharmaceutical Sciences, 16th edition, Osol, A., Ed. (1980), for example in the form of a aqueous or non-aqueous solution, suspension, or emulsion.

The compositions also be entrapped in microcapsules prepared, for example, by coacervation techniques or by interfacial polymerization (for example, hydroxymethylcellulose or gelatin-microcapsules, and poly-[methylmethacrylate] microcapsules, respectively), in colloidal drug delivery systems (for example, liposomes, albumin microspheres, microemulsions, nano-particles, and nanocapsules), or in macroemulsions. Such techniques are discussed, for example, in Remington's Pharmaceutical Sciences, 16th edition, Osol, A., Ed. (1980).

Such formulations may generally be prepared as immediate or sustained-release preparations and can administered by orally or, for example, by injection, subcutaneous implantation, or by implantation at a desired target site. Sustained-release formulations contain anti-MJA antibodies or other MJA-agents, and may contain secondary therapeutic agents, dispersed in a carrier matrix or contained within a reservoir surrounded by a rate controlling membrane or otherwise as desired. In some embodiments the formulation provides a relatively constant level of release; controlled variable release is also possible. The amount of active compound contained within a sustained release formulation depends upon the site of deposit, the rate and expected duration of release and the nature of the condition to be treated or prevented.

Representative examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as discussed by Langer et al., J. Biomed. Mater. Res., 15:167-277 (1981), and Langer, Chem. Tech., 12:98-105 (1982), or poly(vinylalcohol)), polylactides, U.S. Pat. No. 3,773,919; EP 58,481, copolymers of L-glutamic acid and gamma ethyl-L-glutamate, Sidman et al., Biopolymers, 22:547-556 (1983), non-degradable ethylene-vinyl acetate, Langer et al., supra, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid, EP 133,988.

Acceptable carriers, adjuvants, excipients, buffers and diluents are nontoxic to recipients at the dosages and concentrations employed. Representative carriers and buffers include physiological saline solutions, gelatin, sterile water, inert solids, alcohols, natural or synthetic oils, saccharide solutions, glycols, fats, waxes, injectable organic esters such as ethyl oleate or a combination of such materials, phosphate buffer, citrate buffer, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA or glutathione; sugar alcohols such as mannitol or sorbitol; salt-forming counterions such as sodium; or nonionic surfactants such as Tween, Pluronics, or polyethylene glycol (PEG), neutral buffered saline or phosphate buffered saline), and antimicrobial compounds. Representative adjuvants include aluminum hydroxide and other adjuvants discussed elsewhere herein or otherwise as desired.

An anti-MJA therapeutic to be used for in vivo human administration should be sterile. This can be accomplished by filtration through sterile filtration membranes, for example prior to or following lyophilization and reconstitution. The composition can be stored in lyophilized form or in solution. Therapeutic antibody compositions generally are placed into a container having a sterile access port, for example, an intravenous solution bag or vial having a stopper pierceable by a hypodermic injection needle and therefore suitable for use with a hypodermic syringe.

### Routes of Administration

The route of antibody administration can be, for example, orally or by injection or infusion by subcutaneous, intravenous, intraperitoneal, intracerebral, intramuscular, intraocular, intracranial, intraarterial, intranasal, or intralesional routes, by sustained release systems or otherwise as desired. Non-parenteral routes are discussed further, for example, in WO 96/20732. The composition can be administered, for example, continuously by infusion or by bolus injection.

### Dosage Amount

In therapeutic applications, a pharmaceutical composition (e.g., comprising an anti-MJA antibody) is administered to a patient intravenously. A therapeutically effective dose or efficacious dose, i.e., an effective amount, is an amount that will ameliorate one or more of the known parameters that characterize medical conditions caused or mediated by, or otherwise related to, MJA biological activity. Amounts effective for this use will depend upon the patient's plasma level of MJA, the general state of the patient, and combination with other anti-disease drugs or other secondary agents, if any, but generally range from about 10 ng to about 10 g of active agent per dose, with single dosage units of from about 10 ng to 100 mg per patient.

Desirable dosages include 250 μg, 500 μg, 1 mg, 50 mg, 100 mg, 200 mg, 300 mg, 500 mg, 750 mg, 1 g, 1.5 g, 2 g, 3 g, 4 g, 5, 6, and 10 g. In some embodiments, the dose of the antibodies discussed herein produces a tissue or blood concentration or both from about 0.1 μM to 500 mM. Such doses can vary from about 1 to 800 μM, or from about 10 μM to about 500 μM. Desirable doses are, for example, enough antibody to achieve a tissue or blood concentration or both of 10 μM, 15 μM, 20 μM, 25 μM, 50 μM, 75 μM, 100 μM, 150 μM, 200 μM, 250 μM, 300 μM, 400 μM, and 500 μM.

An effective amount of anti-MJA agent to be employed in a pharmaceutical composition will depend, for example, upon the therapeutic objectives, the route of administration, and the condition of the patient. The given dosage is chosen by an individual physician in view of the patient to be treated. Dosage and administration are adjusted to provide sufficient levels of the active moiety or to maintain the desired effect. Additional factors that can be taken into account include the severity of the disease state of the patient, sex, age, and weight of the patient; diet, time and frequency of administration, the severity of any infection or other condition, including complicating conditions, drug combination(s), reaction sensitivities, and tolerance/response to therapy, and other clinical factors. Short acting pharmaceutical compositions are administered daily whereas long acting pharmaceutical compositions are administered every 2, 3 to 4 days, every week, or once every two weeks. Depending on half-life and clearance rate of the particular formulation, the pharmaceutical compositions of the invention are administered once to ten or more times per day. The therapist may titrate, often upward, the dosage and modify the route of administration as appropriate to obtain the desirable therapeutic effect. The progress of therapy can be monitored by conventional assays.

### EXAMPLES

### Example 1

Isolating, Culturing and Storing MJA from Blood.

Equipment and Media:

Incubator equipped with CO2 control, set at 5% CO2.

Cylinder of CO2.

Meter for CO2 cylinder, set at 15 PSI.

Vitacell RPMI-1640 culture medium with 20% fetal calf serum (ATCC).

Crystal Violet 2% aqueous.

Sterile pipettes, 2.0 ml and 10 ml.

Sterile centrifuge tubes, 10 ml or 2 ml.

Automatic pipettor.

Liquid nitrogen or −70 freezer.

Glass slides and cover slips.

Procedure:

Blood was collected from a colitis patient using buffered sodium citrate anticoagulant, and anticoagulated by gently swirling the mixture. 2 ml Vitacell were dispensed into sterile centrifuge tubes. 1 ml of whole blood was added to the tubes then mixed by gentle rotation. The mixtures were incubated at 39° C. in atmosphere of 5% CO2. The culture was checked at 24 and 48 hours. The culture turned color from pink to yellow tint as an indicator of cell growth.

To assay for the presence of MJA, 1 drop of culture (cells and fluid) was placed on a glass slide. 1-2 drops of aqueous crystal violet were added. A cover slip was wet mounted and the edges sealed with permount or fingernail polish then examined under a microscope. Erythrocytes from the blood contained MJA, which appeared as intracellular particles that stained with the crystal violet. MJA was also found in the supernatant fluid. Exemplary photomicrographs of blood/MJA prepared as discussed in this Example can be seen in FIGS. 2 & 3.

To cryopreserve, 10 ml of culture (2:1 Vitacell:blood) was mixed with 10 ml of a 7.5% glycerol in Ringers lactate solution. Cooling was carried out in a refrigerator. Aliquots (about 2 ml) were extracted and placed in 2 ml cryovials then placed in liquid nitrogen.

Two aliquots were retrieved from the liquid nitrogen, and warming was carried out slowly. The thawed samples were placed into further Vitacell medium and grown for 3 days at 37° C. Microscopic review found that the numbers of MJA in the 3-day old cultures had increased relative to the mixture after thawing.

The MJA from culture can also be used otherwise as desired.

### Example 2

Crystal Violet Staining.

Some samples of MJA were stained with crystal violet according to normal procedures except that the sample was stained fresh, unfixed as a wet mount. Crystal violet was applied to samples prepared as discussed in Example 2 as an aqueous solution at 1% or 2% w/v. The wet mount was sealed with finger nail polish. Microscopic examination was carried out promptly, before the sample could significantly degrade. Exemplary photomicrographs of blood/MJA prepared as discussed in this Example can be seen in FIGS. 2 & 3.

### Example 3

Growing MJA in Chicken Eggs

MJA was cultured in fertilized hens eggs, obtained disease free from Oregon State University Poultry Research (Corvallis, Oreg.), as follows.

Specimens:

The sources of MJA were:

Specimen A: Anticoagulated human whole blood containing the microorganism as seen in wet mounts stained with 1% crystal violet in physiologic saline. The blood had been collected from a patient with diarrhea and bronchitis and had been stored in liquid nitrogen for three weeks.

Specimen B: Two 2 ml cryovials prepared as set forth in Example 1 each containing pooled human blood samples that showed MJA on wet mount. The cryovials were stored in liquid nitrogen. For use, the cryovials were removed from the liquid nitrogen and thawed at room temperature (70° F.) for several hours.

Inoculation and Growth:

Six chicken eggs 6 days post-fertilization were used for inoculation, which was performed at room temperature. 6 additional eggs served as controls. The eggs were candled to locate the embryo and to confirm viability. The air sac was found at the blunt end. This end was sterilized with iodine and wiped with propyl alcohol. A Dremel® rotary tool was used to make a small hole in this end, over the air sac. The area was swabbed again with iodine and allowed to dry. 0.5 ml of each of the source specimens were aspirated through a 22 gauge, 1 inch needle into a 3 ml syringe. The needle was inserted along the long axis of the egg through the hole, aimed in front of the embryo. Slow injection of the 0.5 ml inoculant was performed and the needle withdrawn. The inoculation site was wiped with iodine, allowed to dry and sealed with epoxy glue.

Specimen A was inoculated into two eggs, Nos. 1 and 2.

Each cryovial of Specimen B was inoculated into two eggs, Nos. 3 and 4, and Nos. 5 and 6, respectively.

The eggs were incubated at 97° F. for 7 days in an incubator with a water dish. The eggs were rotated frequently, end over end, to inhibit adhesions of the embryo.

Harvesting:

The eggs were refrigerated at 34° F. for 18 hours prior to harvesting. This shrank the blood vessels and reduced blood contamination of fluid chambers. The eggs were washed in propyl alcohol. The area over the air sac was wiped with iodine. The shell was cracked with a blunt object and pieces of shell removed, revealing the shell membrane. An area 1 inch square was uncovered. The shell membrane was pierced and removed revealing the allantoic cavity. The allantoic fluid was greatly increased relative to the control eggs.

This allantoic fluid was aspirated into a 10 ml syringe and then placed in a 15 ml sterile centrifuge tube. 7-8 ml of allantoic fluid were aspirated from eggs Nos. 1 and 2 (from Specimen A; not all the fluid was removed), 7-8 ml of allantoic fluid were aspirated from eggs Nos. 3 and 4 (from Specimen B; not all the fluid was removed), while about 6-8 ml allantoic fluid was removed from each of eggs Nos. 5 and 6 (Specimen B).

The allantoic fluid was cloudy gray. A drop was placed on a microscope slide. One or two drops of crystal violet solution were added. The preparation was cover slipped and sealed with nail polish. Examination under the microscope showed about a 4 times increased density of MJA compared to the specimens prior to inoculation. Since about 0.5 ml of inoculum was injected and 15 ml of allantoic fluid were recovered, it is estimated that the total number of MJA increased about 120 times.

Allantoic fluid from 3 eggs was placed in 30 numbered, sterile cryovials as follows:

Egg No. 2 (Specimen A): 0.5 ml in cryovials 1-8.

Egg No. 6 (Specimen B): 0.5 ml placed in cryovials 9-20.

Egg No. 5 (Specimen B): 0.5 ml placed in cryovials 21-30.

The cryovials were refrigerated 2 hours at 34° F. and plunged in liquid nitrogen.

15 ml test tubes containing allantoic fluid from eggs Nos. 4, 5 and 6 were also placed straight into liquid nitrogen. Four cryovials each containing 0.5 ml allantoic fluid from egg No. 4 were fixed in 1.5 ml glutaraldehyde for electron microscopy.

Three cryovials containing pieces of yolk sac were also placed in liquid nitrogen. The remaining content of the egg (the yolk) was decanted into a sterile Petri dish after first separating the yolk sac from the embryo. The yolk sac was isolated in the Petri dish. Its membrane was incised and the membrane removed and fixed in 10% formalin.

The remainder of the egg was discarded.

### Example 4

Histology on Yolk Sacs from Example 4.

Yolk sacs were isolated from every egg from Example 4 to provide 6 infected samples and 6 control samples. The specimens were embedded in paraffin and sections from each were H&E stained and Steiner stained. The Steiner stain on the 6 infected eggs showed MJA in large numbers. The infected eggs showed MJA in blood, in RBCs, in blood vessels, in yolk sac cells and in the yolk. A photomicrograph of a yolk sac prepared as discussed in this Example can be seen as FIG. 16. The H&E stains were inconclusive as to the presence of MJA but were helpful in identifying structures in the specimens. Steiner stain on the 6 control eggs showed no MJA.

### Example 5

Re-Growing MJA in Chicken Eggs.

Two cryovials containing pure allantoic fluid infected with MJA from Example 3 were thawed at room temperature then used to infect new fertilized chicken eggs according to the procedure set forth in Example 3. The new chicken eggs grew MJA, as seen by microscopic examination of the allantoic fluid.

### Example 6

Determination of Antigenicity of MJA.

To determine if four colitis patients infected with MJA (as determined by silver staining) carried antibodies against MJA, tissue sections of the colon from the four patients were incubated with the patient's own serum under routine conditions. Mouse anti-human gamma globulin antibodies were applied to the tissue sections under routine conditions. The presence of binding was determined using the immuno-peroxidase method. Binding of the mouse antibodies was observed at the same sites where MJA was found by silver staining sections of the same biopsies. This indicates that MJA is antigenic.

### Example 7

Growing MJA of Solid Media

Fresh human blood from a colitis patient was smeared on a slide and a wet-mount stained with 2% crystal violet. The sample contained large numbers of MJA. FIG. 2. Further fresh blood from the patient was spread with a sterile loop on culture plates containing media. Two media (PML Microbiologicals, Willsonville, Oreg.) were used: 1) TSA with 5% sheep blood, or 2) Chocolate agar with enrichment. The plates incubated for 12 days at 37° C. There was no growth on the culture plates.

This indicates that MJA does not grow on these two commonly used solid culture media.

### Example 8

Treatment of Patents

Patient 1, male age 62.

Clinical diagnosis: Three episodes of fever. Renal failure with the last episode. Clinical diagnosis: thrombotic thrombocytopenic purpura.

Inventor's further diagnosis: Vessels blocked by MJA.

Treatment: Had upper and lower colonoscopy. Biopsy found gastritis and colitis with precancerous polyps. Administered doxycycline 100 mg bid for 10 days.

Results: Last seen 6 years later, no recurrence.

Patient 2, female age 82.

Clinical diagnosis: Three weeks with chronic bronchitis and pneumonia. She had chronic constipation and intermittent diarrhea.

Inventor's further diagnosis: Bronchial and bowel involvement of MJA. Colitis and bronchitis.

Treatment: Doxycyline 100 mg bid for 7 days.

Results: Generally recovered after 5 days. Still constipation, some colon complaints. Seborrheic keratosis gone from face and neck.

Patient 3, male age 35.

Clinical diagnosis: Flu with muscle aches and abdominal nausea. Weak, obtunded and chills.

Inventor's further diagnosis: Flu die to MJA. Blood wet prep showed MJA.

Treatment: Doxycyline 100 mg bid for 10 days.

Results: Well in 3 days. No recurrence. EM showed degenerate forms of MJA in red blood cells.

Patient 4, female age 30.

Clinical diagnosis: Severe fever, flu syndrome, chills, vomiting, abdominal pain and muscle aches.

Inventor's further diagnosis: “Flu” due to MJA. Her husband is diabetic. She has had a previous miscarriage.

Treatment: Doxycyline 100 mg bid for 10 days.

Results: Recovered fully. Last seen in 9^{th} month of normal pregnancy.

Patient 5, female age 54.

Clinical diagnosis: History of ulcerative colitis since her late 20's.

Inventor's further diagnosis: MJA infection. Chronic colitis.

Treatment: Doxycyline 100 mg bid for 3 days.

Results: Pain in right shoulder gone. Seborrheic keratosis gone. Decreased nasal mucus.

### Example 9

Selection of Antigenic Peptides

Antigenic peptides are derived from the amino acid sequence of MJA based on analyses of likely antigen-containing regions. Design of antigen peptides (about 20 amino acids in length) for antibody generation is performed using basic techniques, including BLAST methods of peptide analysis to determine regions comprising (1) specificity to MJA, and (2) antigenicity. With respect to specificity, parameters that mitigate against the use of a particular peptide include the presence of 6 or more contiguous amino acids with sequence identity to protein(s) other than MJA, the presence of sites of posttranslational modification, including phosphorylation and glycosylation, and highly hydrophobic sequences, which could indicate potential in situ localization within the plasma membrane.

In alternative approaches, the full MJA protein, or nucleic acid sequences encoding MJA or desired portions thereof, are used.

### Example 10

Preparation of Antibody Dilutions

The purpose of this protocol is to dilute antibodies in solution. Materials include Tris-HCL Buffer with carrier protein and 0.015 M NaN₃ (Dako Antibody Diluent #S0809 (DAKO, Carpentaria, Calif.); vials containing the antibodies discussed above or commercial antibodies against MJA; pipetmen and disposable tips; container of chopped ice; 12 ml Dako reagent tubes; and, reagent tube rack.

The procedure is a) calculate proportions of antibody and diluent according to desired concentrations and volume requirements; b) label reagent tubes and place in rack; c) pipette needed volume of diluent into tube(s); d) place vials of antibodies into ice; e) invert and/or flick antibody vial(s) 3 or 4 times to insure suspension; f) pipette required volume of antibody(s) into corresponding diluent volumes; and, g) mix gently.

From the foregoing, it will be appreciated that, although specific embodiments have been discussed herein for purposes of illustration, various modifications may be made without deviating from the spirit and scope of the disclosure. Accordingly, the systems and methods, etc., include such modifications as well as all permutations and combinations of the subject matter set forth herein and is not limited except as by the appended claims.

1. A labeled image wherein the image comprises MJA and a label specifically identifying the image as depicting MJA.

2. The labeled image of claim 1 wherein the image further comprises indicators indicating at least one location of MJA in the image.

3. The labeled image of claim 1 wherein the image is maintained in a computer memory.

4-6 cancelled.

7. A computer memory configured to specifically identify data therein as representing MJA.

8. The computer memory of claim 7 wherein the data comprises an image of MJA.

9. The computer memory of claim 8 wherein the data further comprises at least one label specifically identifying the image as MJA.

10-13 cancelled.

14. A composition comprising an isolated antibody specific substantially only for MJA.

15-20 cancelled.

21. A composition configured for use with an animal, the composition comprising a pharmaceutical amount of an antibody suitable for administration to the animal and specific substantially only for MJA, and at least one of a pharmaceutically acceptable carrier, adjuvant, excipient, buffer and diluent.

22-34 cancelled.

35. A kit for the detection of MJA in a sample comprising:
a) at least one antibody specific substantially only for MJA;
b) at least one of a reagent and a device for detecting the antibody; and,
c) a label stating that the kit is to be used for the detection of MJA.

36. An assay for the detection of MJA in a sample, comprising:
a) providing an anti-MJA antibody,
b) contacting the anti-MJA antibody with the sample under conditions suitable and for a time sufficient for the anti-MJA antibody to bind to MJA present in the sample, to provide an antibody-bound MJA, and
c) detecting the antibody-bound MJA, and therefrom determining whether the sample contains MJA.

37-41 cancelled.

42. An assay for the detection of MJA in a sample, comprising:
a) providing an anti-idiotypic anti-MJA-antibody antibody,
b) contacting the anti-idiotypic anti-MJA-antibody with the sample under conditions suitable and for a time sufficient for the anti-idiotypic anti-MJA-antibody to bind to anti-MJA-antibody present in the sample, to provide an antibody-bound anti-MJA-antibody, and
c) detecting the antibody-bound anti-MJA-antibody, and therefrom determining whether the sample contains MJA.

43-47 cancelled.

48. A pharmaceutical amount of at least one of an isolated humanized or fully human monoclonal antibody specific substantially only for MJA for use in the manufacture of a medicament for inhibiting, preventing or treating MJA in an animal.

49-50 cancelled.

51. A method of treating an animal, comprising determining that the animal has a putative MJA infection, then selecting a pharmaceutical composition comprising an anti-MJA drug, then administering the anti-MJA drug composition to the animal.

52-54 cancelled.

55. A method of reducing an amount of MJA in an animal comprising administering an effective amount of a selected anti-MJA drug to the animal in an amount and for a time sufficient to reduce the amount of the MJA infection.

56. A method of reducing at least symptom associated with an MJA infection in an animal comprising administering an effective amount of a selected anti-MJA drug to the animal in an amount and for a time sufficient to reduce the at least symptom associated with the MJA infection.

57-85 cancelled.
